(19) United States
(12) Patent Application Publication
(10) Pub. No.: US 2026/0248633 A1
(43) Pub. Date: Aug. 27, 2026

(54) AN ABSORBENT INSERT OSTOMY DEVICE FOR INSERTION THROUGH AN OSTOMY OF A HUMAN BEING

(71) Applicant: AMPA MEDICAL APS, København N (DK)

(72) Inventors: Marie Filippson PARSLOV, København N (DK); Cecilie AMMITZBØLL, København N (DK)

(73) Assignee: AMPA MEDICAL APS, København N (DK)

(21) Appl. No.: 18/874,723

(22) PCT Filed: Jun. 15, 2023
(86) PCT No.: PCT/EP2023/066108

§ 371 Date: Dec. 13, 2024

(30) Foreign Application Priority Data
Jun. 16, 2022 (DK) PA202270324

(51) Int. Cl. A61F 5/44; A61F 13/15; A61F 13/53

(52) U.S. Cl. CPC A61F 5/4401; A61F 13/15; A61F 2013/15008; A61F 2013/15471; A61F 2013/530036

(57) ABSTRACT

Absorbing closures for liquid and semi-liquid intestinal wastes from intestinal openings that are ideally free from the physical and psychological drawbacks from stoma bags. An absorbent insert ostomy device for insertion through an ostomy of a subject, the device being configured to absorb and/or collect intestinal waste inside the intestines of the subject.

### TECHNICAL FIELD

The invention relates to an absorbent insert ostomy device for insertion through an ostomy of a human being, the device being configured to absorb intestinal fluid and waste inside the intestines of the human being.

### BACKGROUND ART

An ileostomy is an artificial intestinal opening where the large intestine (colon) and sometimes part of the small intestine is surgically removed due to illness or trauma and the remaining part of the small intestine (jejunum or ileum) is redirected through the abdominal wall and sutured to the skin, constituting the ileostomy. Hereafter the intestinal waste is emptied through the ileostomy. Ileostomies do not have a sphincter to control voluntary excretion of intestinal waste and therefore the ileostomy is incontinent with continuous and uncontrollable excretion of intestinal waste. Beside ileostomies, other ostomies can be created, such as a colostomy (emptying waste from colon) or an urostomy (emptying urine). The ileostomy term, is in general covering both ileostomies (ileum) and jejunostomies (jejunum). All the above-mentioned localized ostomies may generally be known as ostomies.

The intestinal waste from all kinds of ostomies is collected in a stoma bag attached to a person's stomach by an adhesive plate. The stoma bag was invented in the 1950's and has since then been used to collect the intestinal waste from an ostomy. However, the stoma bag has considerable disadvantages.

First of all, the changed body image that comes with having a bag with intestinal waste hanging from the abdomen is a psychological strain for most people.

Secondly, since a person with an ostomy lacks the ability to hold back intestinal waste and intestinal gases, and the excretion of intestinal waste and intestinal gases into the stoma bag cause considerably noise and a characteristic smell during bowel movement, the user is often considerably socially handicapped-even when stoma bags are used properly.

Thirdly, the adhesively attached stoma bags moreover have the disadvantage that the weight of the more or less filled bag causes discomfort and may result in the bag becoming involuntarily detached from the user's skin, resulting in leakage of intestinal waste. In addition, natural folding in the skin surrounding the stoma can cause difficulties in keeping the adhesive plate tight from leakage. The constant worry of leakage is a mental burden that almost all users of stoma bags live with. Moreover, users of stoma bags often get skin complications requiring medical treatment, due to skin contact with intestinal waste and the aggressive adhesive.

Finally, physical exercise is complicated by the external storage of intestinal waste in a bag. Upon pushing, squeezing, or clamping the bag there is a risk of leakage. In general, stoma bags require a protruding stoma, i.e. a stoma that protrudes a couple of centimetres from the skin. A protruding stoma works as a kind of faucet, such that the intestinal waste is guided into the bag, while minimizing the contact with the surrounding skin. However, this is not always successfully achieved and this leads to skin ulcerations. Furthermore, the protruding stoma may add to the mental burden of the user of the stoma bag.

People with ostomies demand alternative solutions to the stoma bag. So far, there are no functional alternatives on the market. Existing solutions that are designed for withholding solid stool in a colostomy (stoma of the large intestine) are not suited for use in an ileostomy because of the large volumes of liquid content that is produced in the ileum. This makes it difficult to seal the ileum properly. For the majority of people with an ileostomy, the intestinal waste is liquid and the volume is 500-1000 millilitre per day.

There is thus a need of another type of system to collect the liquid of the intestine while also forming a closure hereof. In particular, there is a need of a closure that is suited for the small intestine (ileum) and form a closure hereof, and with the ability to retain the majority of that liquid.

### SUMMARY OF THE INVENTION

The present invention solves the above problems by providing an absorbent insert ostomy device for insertion through an ostomy of a human being. The absorbent insert ostomy device provides a device which is able to absorb liquid in the intestine while forming a closure hereof, and with the ability to retain the majority of that liquid, while being withdrawn from the intestine. In particular, the present device provides the needed closure that is suited for placement in the small intestine (ileum) and form a closure hereof, and with the ability to retain the majority of that liquid, while being withdrawn from the ileum.

Hence, disclosed herein in a first aspect is an absorbent insert ostomy device for insertion through an ostomy of a human being, the device being configured to absorb intestinal fluid and waste inside the intestines of the human being, the absorbent insert ostomy device comprising: an expandable elongated body extending along a longitudinal direction of the absorbent insert ostomy device and having a stoma end and an oral end spaced from the stoma end along the longitudinal direction, wherein the expandable elongated body comprises one or more absorbent materials; one or more withdrawal means attached to the stoma end of the expandable elongated body; and one or more compression means configured to maintain the expandable elongated body in a compressed state, having a first diameter along the longitudinal direction, prior to insertion of the absorbent insert ostomy device through the ostomy of the human being; wherein the expandable elongated body is configured to expand radially to a non-compressed state, having a second diameter along the longitudinal direction, when released from the one or more compression means upon insertion into the intestines of the human being through the ostomy; wherein the second diameter is larger than the first diameter; and wherein the expandable elongated body comprising the absorbent material is further configured to be pulled out of the intestines of the human being by means of the one or more withdrawal means.

The present disclosure therefore relates to an absorbent insert ostomy device suitable for insertion through a stoma of a subject such as a human being. The wording “through a stoma” may also be worded as “into a stoma” depending on how the stoma is made and how far into the opening the device protrudes. The device will enter into the stoma opening (the ostomy) and be positioned within the intestines (in case of e.g. an ileostomy). The device may slightly protrude outside the stoma opening. Therefore, the device could both be said to be suitable for insertion through a stoma of a subject such as a human being and/or suitable for insertion into a stoma of a subject such as a human being.

A stoma may herein be limited to an artificially created opening in the body. An example of a natural stoma is the anus, and an example of an artificial stoma is an ileostomy. Behind the body opening is located a body canal such as the rectum, the colon, or the ileum. In one or more embodiments, the stoma is an opening on the abdomen that can be connected to either the digestive or urinary system to allow waste (urine or faeces) to be diverted out of the body. In one or more embodiments, the stoma is an ileostomy.

In other words, the present invention provides a disposable absorbing closure for liquid and semi-liquid intestinal contents from artificial intestinal openings. The invention is free from the physical and psychological drawbacks from stoma bags and surgical solutions. The invention consists of an absorbent organ, shaped and constructed so that the organ expands in the intestine upon absorption to a radial diameter, but are easily extracted through a stoma opening while retaining absorbed intestinal content. The invention is capable of absorbing large amounts of intestinal content. However, the invention can also be used in a natural intestinal opening.

The absorbent insert ostomy device is configured to be placed inside such a body canal and form a closure of the body canal. In particular, the absorbent insert ostomy device as disclosed herein is suited for insertion through an ostomy and for placement inside e.g. the ileum of the subject. The absorbent insert ostomy device comprises an absorbent material for absorbing liquids, in particular intestinal waste of the subject.

The absorbent insert ostomy device comprises one or more withdrawal means e.g. attached to the elongated body at one end of said body, such that the device can be withdrawn with ease from the subject using said withdrawal means. The withdrawal means may be a string or a cord such that the insert device may be removed from the subject by pulling the string/cord. As an example, the string or cord may be made of cotton, nylon, silicone, rubber, polyester, and/or combinations thereof. However, other materials for the string/cord could be used. Preferably, the withdrawal means is configured to protrude from the stoma, when the insert device is placed inside the body canal, such that the subject can pull the withdrawal means to remove the insert device from the body canal. Any of the embodiments of the insert device as described herein may be provided with the withdrawal means. To ensure the absorbent device stays in place, a button or similar may be attached to the end of the withdrawal means.

In general, tampon-like closures have the drawback that they typically have a small absorption capacity. If used in the intestine, this may result in intestinal waste not being able to be absorbed by the closure and thereby leak or accumulate behind the closure, which potentially leads to an increased pressure on the intestinal wall. The increased pressure can result in dilation of the intestine, or it can result in the closure being pushed out of the intestine, both of which are undesirable situations. A further challenge with a tampon-like closure is that the diameter of the stoma is smaller than the elastic diameter of the intestine, whereby there is a risk of squeezing the closure upon pulling it out through the stoma, whereby some of the liquid intestinal waste is spilled. For these reasons, no tampon-like solution has ever been commercialized for usage in ostomies. However, as demonstrated herein, the disclosure of the present invention has nicely solved these problems. Hence, disclosed herein in a second aspect is an absorbent insert ostomy device for insertion through an ostomy of a human being, the device being configured to absorb intestinal fluid and waste inside the intestines of the human being, the absorbent insert ostomy device comprising: an expandable elongated body extending along a longitudinal direction of the absorbent insert ostomy device and having a stoma end and an oral end spaced from the stoma end along the longitudinal direction, wherein the expandable elongated body comprises one or more absorbent materials, wherein the one or more absorbent materials at least comprises carboxymethyl cellulose; one or more withdrawal means attached to the stoma end of the expandable elongated body; and one or more compression means configured to maintain the expandable elongated body in a compressed state, having a first diameter along the longitudinal direction, prior to insertion of the absorbent insert ostomy device through the ostomy of the human being; wherein the expandable elongated body is configured to expand radially to a non-compressed state, having a second diameter along the longitudinal direction, when released from the one or more compression means upon insertion into the intestines of the human being through the ostomy; wherein the second diameter is larger than the first diameter; and wherein the expandable elongated body comprising the absorbent material is further configured to be pulled out of the intestines of the human being by means of the one or more withdrawal means.

Carboxymethyl cellulose (CMC) or cellulose gum is a cellulose derivative with carboxymethyl groups (—CH₂COOH) bound to some of the hydroxyl groups of the glucopyranose monomers that make up the cellulose backbone. It is often used as its sodium salt, sodium carboxymethyl cellulose.

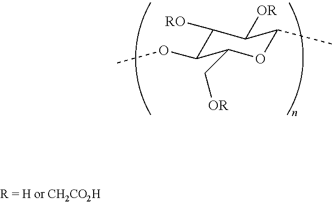

### Schematic Drawing of CMC—for Illustrative Purposes.

CMC is normally used in food as a viscosity modifier or thickener, and to stabilize emulsions in various products including ice cream. However, it has also found use in a constituent of many non-food products, such as toothpaste, laxatives, diet pills, water-based paints, detergents, textile sizing, reusable heat packs, various paper products, and also in leather crafting to help burnish the edges. It is used primarily because it has high viscosity, is nontoxic, and is generally considered to be hypoallergenic as the major source fibre is either softwood pulp or cotton linter.

Further, knitted fabric made of cellulose (e.g. cotton or viscose rayon) has also been used in various medical applications when converted into CMC, e.g. as a device for epistaxis where a poly-vinyl chloride (PVC) balloon is covered by CMC knitted fabric reinforced by nylon. The device is soaked in water to form a gel, this is inserted into the nose and the balloon inflated. The combination of the inflated balloon and the therapeutic effect of the CMC stops the bleeding. Such fabric has also been used as a dressing in surgical procedures. Such fabric may also be referred to as CMC Gelling Fibre Dressing, which as disclosed herein is referring to a sterile, single-use dressing which composed of sodium carboxymethylcellulose fibres for wounds with moderate to heavy exudate. The fibre in the dressing rapidly swells after absorbing the wound exudate to form a cohesive transparent gel. The gelling fibre can absorb and lock the exudate, and the bacteria and harmful substances in the exudate are also locked in the dressing, reducing the risk of wound infection and maceration, and maintaining the optimal moist healing environment.

By using CMC as at least part of the one or more absorbent materials, enables the device to enter the ostomy opening, expand radially and longitudinally to a configuration tailored to the dimension of the intestine while absorbing intestinal waste and retain the intestinal waste while being withdrawn through the stoma. This also means that the device is configured to absorb and/or collect intestinal waste inside the intestine, such as the ileum, of the subject, and comprises an expandable elongated body having an initial length and comprising an absorbent material, and withdrawal means attached to the elongated body, wherein the expandable elongated body is configured to expand radially upon insertion into the intestine through the stoma to form a leak-tight closure of the stoma.

This means that the overall initial diameter (as seen in a cross section through the longitudinal axis of the device) of the device (also according to the first aspect) prior to insertion is smaller than or equal to the overall diameter (as seen in a cross section through the longitudinal axis of the device) of the device after it has been inserted into the stoma and released. This can be done by using various compression means, such as utilising an insertion sleeve or applicator surrounding the absorbent material prior to insertion or by inclosing the absorbent material in foil or plastic that is dissolved when the device is inserted in the stoma. The absorbent material hereby undergoes a change from a compressed state in which it has a size that makes it able to enter the stoma to a non-compressed state (i.e. the material expands), such that it may make a (tight) closure of the stoma e.g. by being placed against the inside walls of the ilium or by the intestinal mucosa folding tightly around the device.

It is important to choose an absorbent material, which is not irritating towards the mucus barrier of the intestines. Further, the material may not perforate or in any other way cause mechanical damage to the intestines, not may the chosen material dry out the mucus barrier of the intestines. It is further important that the material is chosen from a material not sticking to the mucus barrier. Preferably the material should also expand when absorbing liquid to make a tight barrier abutting the mucus barrier of the intestines.

In these regards, CMC is a very suited material, as CMC is very soft both in dry and wet state. When the CMC gets wet, due to wetting from the mucosa and absorption of feces, it is converted into a cohesive gel. CMC fibres have a natural ‘swelling’ when it comes into contact with liquid. Further, CMC has a relatively high degree of ‘retention’, hereby holding on to the stool even when the device/elongated body is subjected to a slight pressure. Even further, as shown in the examples, CMC absorbs about 34 times its own weight in small intestine feces, which makes it especially suited for the purpose as disclosed herein. Lastly, CMC is known not to adhere to a wet mucosa/mucus barrier, such as the intestinal mucosa.

A CMC material as disclosed herein preferably has the following parameters:
A mass per unit area (measured according to ISO 9073-1:1989) 120-180 g/m²
A liquid absorption (measured according to EN 13726-1:2002) of at least 15 g per 100 cm².
A tensile strength in its dry state (measured according to ISO 13934-1:2013) of at least 3 Newton per centimetre.
A tensile strength in its wet state (measured according to ISO 13934-1:2013) of at least 0.2 Newton per centimetre.
A loss on drying (measured according to JYWN-QM-P02-SOP162) of no more than 25%.
A heavy metal content (measured according to JYWN-QM-P02-SOP162) of no more than 20 microgram per gram.

Disclosed herein in a third aspect is a use of an absorbent insert ostomy device according to any of the first and second aspect to collect intestinal fluid and waste from the intestines of a subject.

Disclosed herein in a fourth aspect is a use of an absorbent insert ostomy device according to any of the first and second aspect for insertion through an ostomy of a human being.

Disclosed herein in a fifth aspect is a method for collecting intestinal waste from the intestines of a subject, the method comprising the steps of:
inserting the absorbent insert ostomy device according to the first and second aspect through the ostomy of a subject, hereby placing the device inside the intestines of the subject;
letting the absorbent material absorb intestinal waste from the subject; and
withdrawing the absorbent insert ostomy device from the subject by use of the one or more withdrawal means.

Disclosed herein in a sixth aspect is a use of modified cellulose for producing an absorbent insert ostomy device according to any of the first or second aspect.

Disclosed herein in a seventh aspect is a use of modified cellulose as an absorbent material in an absorbent insert ostomy device.

Disclosed herein in an eighth aspect is a use of modified cellulose for absorbing intestinal fluid and waste inside the intestines of the human being.

The aspects, as disclosed above, all have the benefits that they completely avoid the use of a stoma bag to collect intestinal waste. As previously described, stoma bags are often associated with discomfort and psychological strain of the wearer. The presently disclosed is configured for use inside the intestine, e.g. the ileum, of the wearer. Hence, it is not visible from the outside and thereby presents a more discreet solution to the user. Furthermore, the presently disclosed device does not require a protruding stoma, as is the case when using a stoma bag. Hence, the surgical procedure in relation to artificially created stomas can be changed in the future, such that the stoma is level with the skin of the person.

Additionally, the presently disclosed aspects solve the problem of how to withdraw an absorbent closure holding an amount of absorbed liquid content from a body canal out through a stoma or natural intestinal opening. This is problematic as natural or artificial intestinal openings may have a smaller diameter than the body canal, which makes it challenging to withdraw an absorbent closure through the opening, without squeezing the closure and thereby spilling some of the absorbed intestinal waste inside the body canal, e.g. intestine. The applicant has solved these challenges by providing an insert device that can be described by (at least) two diameters: a first diameter that could preferably be equal to or less than the diameter of the stoma of the subject, and a second diameter that could preferably approximately equal the diameter of the body canal, wherein the insert device is intended to be placed. Prior to insertion through the stoma, the absorbent insert ostomy device is compressed to a size such that it can be inserted through the stoma. After insertion and removal of the compressing force, the insert device preferably expands radially to largely conform to the diameter of the body canal behind the stoma, wherein it is placed, in order to form a closure of said body canal. Hence, subsequent to insertion, the insert device preferably expands to obtain the second diameter. However, even after expansion and absorbance of intestinal waste, the insert device is still able to be withdrawn from the body canal out through the stoma while retaining (ideally) all of the absorbed liquid content, or most of the liquid content. This ability is preferably achieved by configuring the insert device such that it conforms to the diameter of the stoma, e.g. achieved by the first diameter, when pulled out of the body canal by means of the withdrawal means.

Disclosed in an ninth aspect is an absorbent insert ostomy device for insertion through an ostomy of a human being, the device being configured to absorb intestinal fluid and waste inside the intestines of the human being and to form a leak-tight closure of the ostomy, the absorbent insert ostomy device comprising: an expandable elongated body extending along a first longitudinal axis and having a stoma end and an oral end spaced from the stoma end along the first longitudinal axis, wherein the expandable elongated body comprises one or more absorbent materials; one or more withdrawal means attached to the stoma end of the expandable elongated body; and one or more compression means configured to maintain the expandable elongated body in a compressed state, having a first diameter along the first longitudinal axis, prior to insertion of the absorbent insert ostomy device through the ostomy of the human being; wherein the expandable elongated body is configured to expand radially to a non-compressed state, having a second diameter along the first longitudinal axis, when released from the one or more compression means upon insertion into the intestines of the human being through the ostomy; wherein the second diameter is larger than the first diameter, the first diameter being equal to or less than the diameter of the ostomy of the human being and the second diameter being equal to or larger than the diameter of the intestines of the human being; wherein the expandable elongated body comprising the absorbent material is further configured to conform to a third diameter along a second axis when the absorbent insert ostomy device is pulled out of the intestines of the human being by means of the one or more withdrawal means; wherein the second axis is an axis extending from the one or more withdrawal means through the ostomy, the second axis being perpendicular to a transverse plane of the ostomy; and wherein the third diameter is substantially equal to the diameter of the ostomy.

### BRIEF DESCRIPTION OF THE DRAWINGS

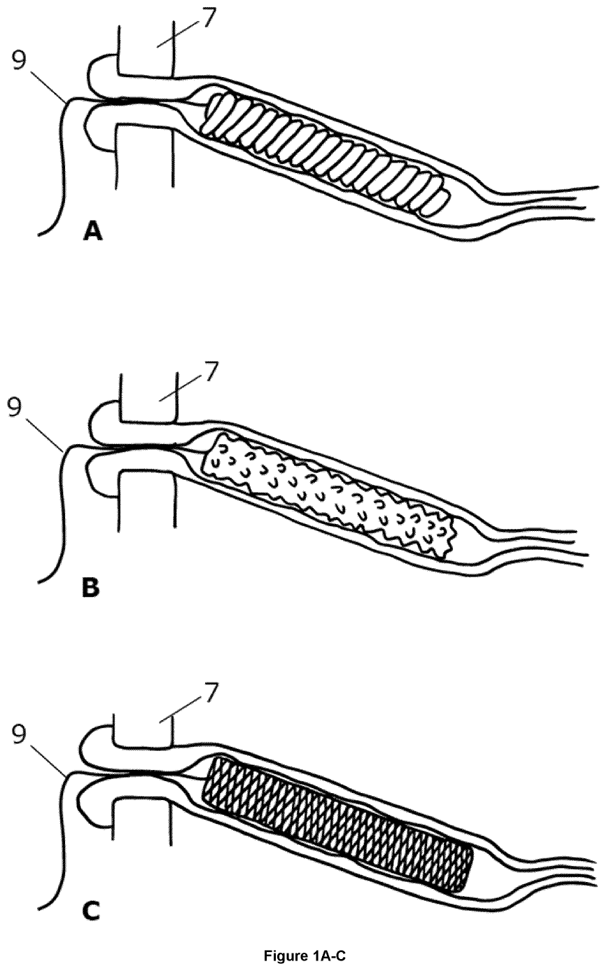

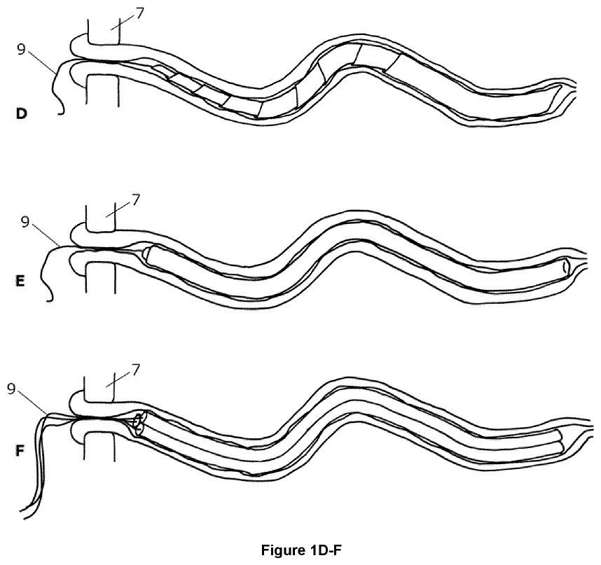

FIG. 1A shows the absorbent insert ostomy device according to an embodiment, wherein the elongated body is curled around a longitudinal axis of the body to form a helix comprising a plurality of turns.

FIG. 1B shows the absorbent insert ostomy device according to an embodiment, wherein the elongated body comprises a plurality of flexible protrusions extending radially from the body, such that the elongated body resembles a brush.

FIG. 1C shows the absorbent insert ostomy device according to an embodiment, wherein the elongated body of the insert device is provided as a flexible tube, which is preferably partly or entirely filled with the one or more absorbent materials.

FIG. 1D shows the absorbent insert ostomy device according to an embodiment, wherein the elongated body of the insert device is formed from a sheet or a strip, which is rolled into an elongated conical shape.

FIG. 1E shows the absorbent insert ostomy device according to an embodiment, wherein the elongated body of the insert device is formed from a tube having a closed end, wherein the tube is folded one or more times inside itself.

FIG. 1F shows the absorbent insert ostomy device according to an embodiment, wherein the elongated body of the insert device is formed from a plurality of cylinders, each cylinder comprising an absorbent material.

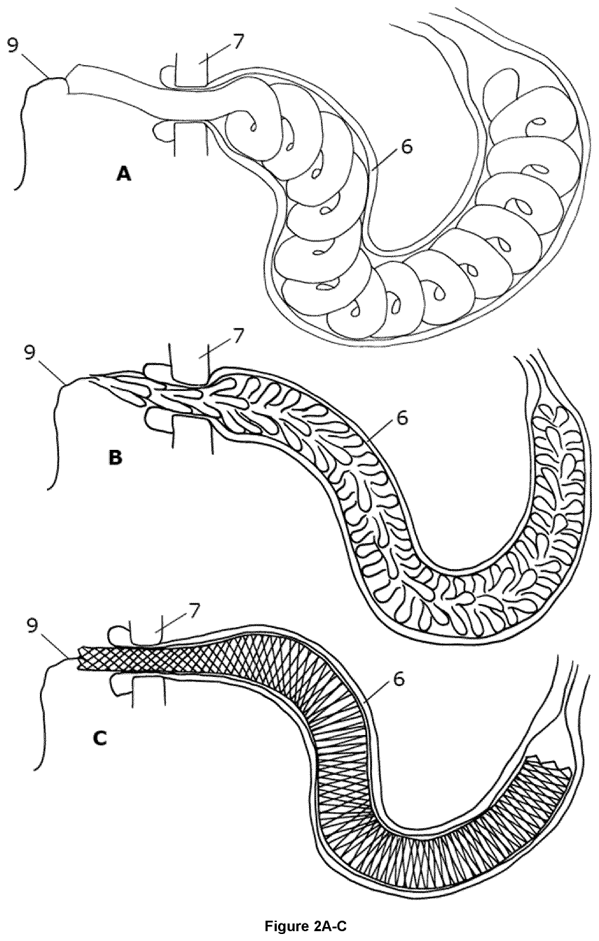

FIGS. 2A-2F shows a non-compressed state of the insert device according to the corresponding (compressed) embodiment of FIG. 1A-1F.

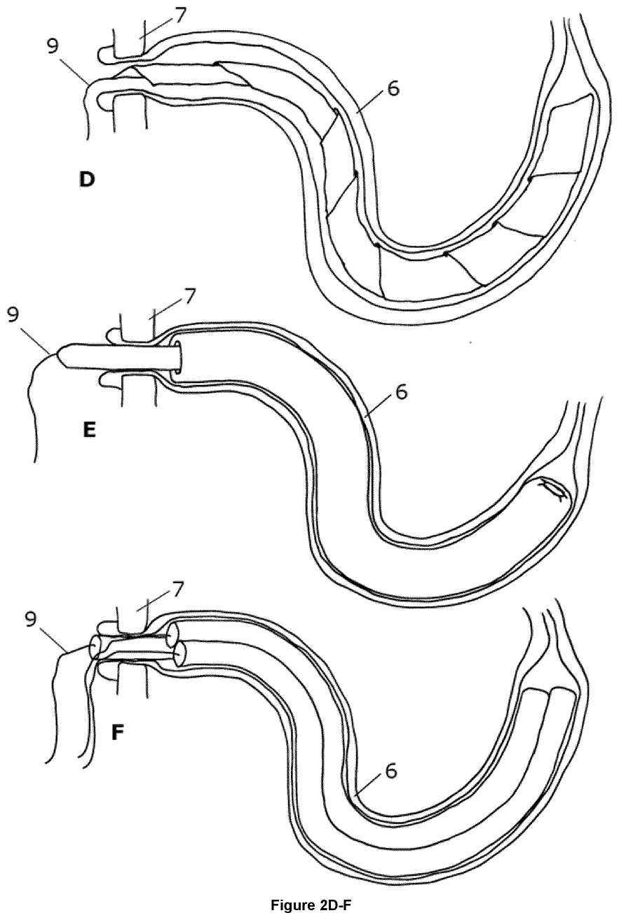

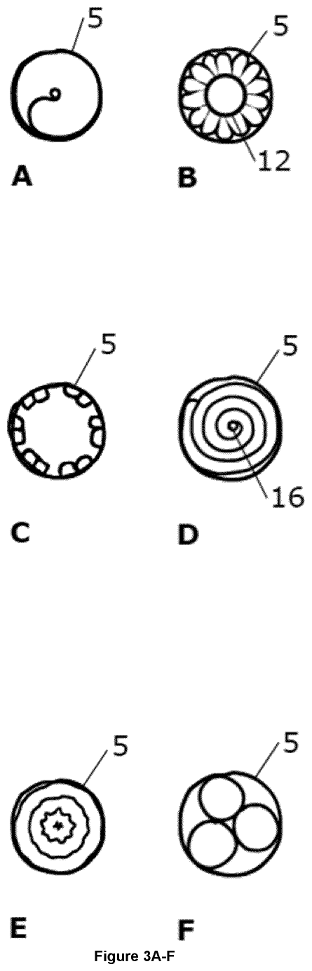

FIGS. 3A-3F shows cross-sections of the embodiments shown in FIGS. 1A-1F. These cross-sections correspond to the compressed state of the insert device.

FIGS. 4A-4F shows cross-sections of the embodiments shown in FIGS. 2A-2F. These cross-sections correspond to the non-compressed state of the insert device.

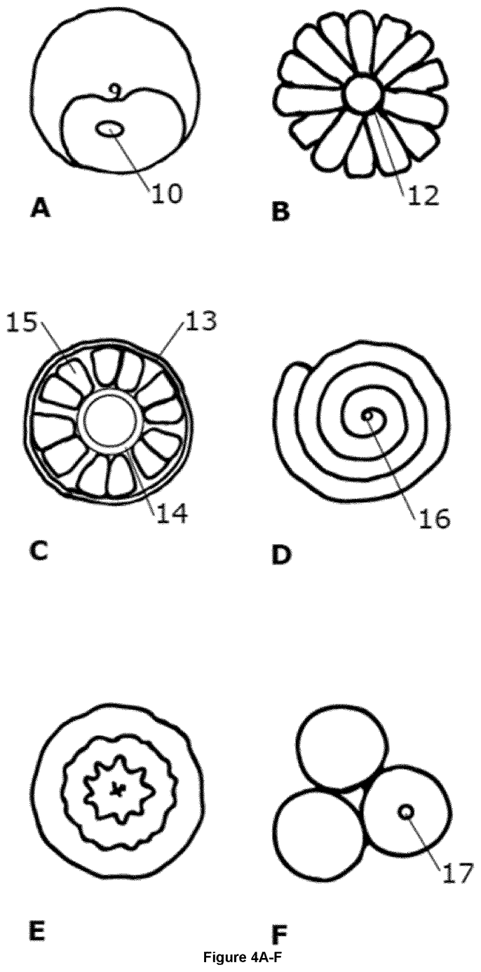

FIG. 5A shows the absorbent insert ostomy device according to an embodiment, wherein the elongated body is formed from a sheet that is folded to form a wave-like pattern of crests and troughs.

FIG. 5B shows the absorbent insert ostomy device according to an embodiment, wherein the elongated body constitutes a cylinder that is folded one or more times.

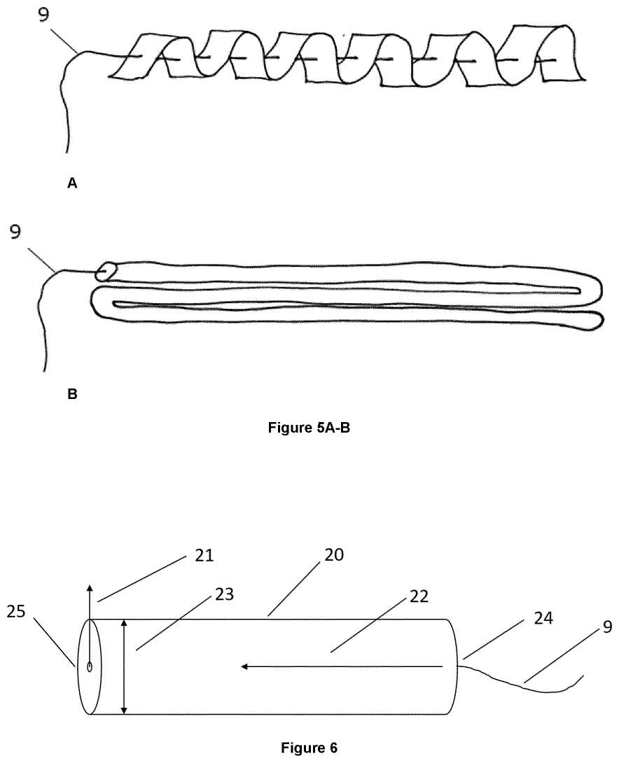

FIG. 6 shows directions of the expandable elongated body as disclosed herein.

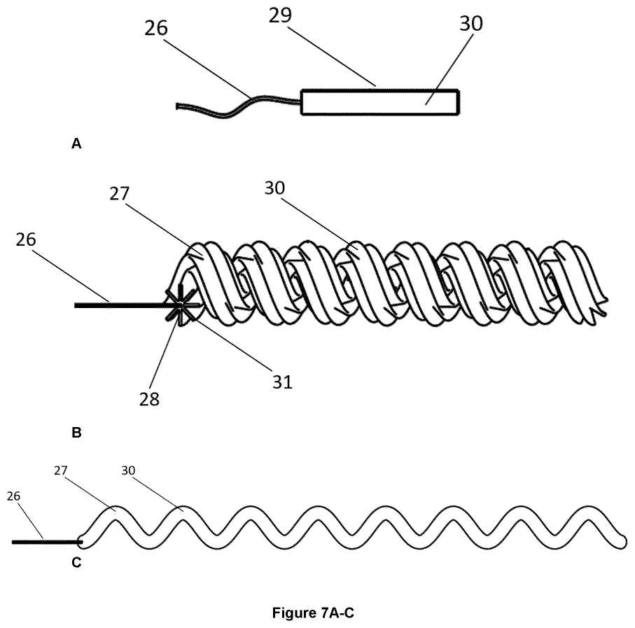

FIG. 7A shows the absorbent insert ostomy device according to an embodiment, wherein the elongated body is in the compressed state.

FIG. 7B shows the absorbent insert ostomy device according to an embodiment, wherein the elongated body is curled around a longitudinal axis of the body to form a helix comprising a plurality of turns and wherein the body further comprises a plurality of flexible protrusions extending radially from the body.

FIG. 7C shows the absorbent insert ostomy device according to an embodiment after withdrawal of the device from the subject.

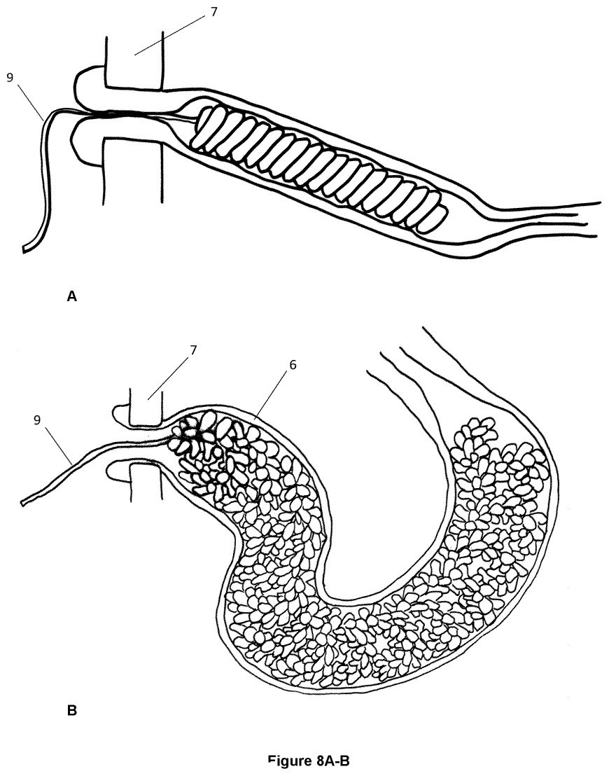

FIG. 8A shows the absorbent insert ostomy device according to an embodiment, wherein the elongated body is curled around a longitudinal axis of the body to form a helix comprising a plurality of turns and wherein the elongated body comprises a plurality of flexible protrusions extending radially from the body, such that the elongated body further resembles a brush.

FIG. 8B show a non-compressed state of the insert device according to the corresponding (compressed) embodiment of FIG. 8A.

### DETAILED DESCRIPTION OF THE INVENTION

Unless otherwise defined, all terms used herein (including technical and scientific terms) have the same meaning as commonly understood by those skilled in the art to which this invention pertains. It will be further understood that terms, such as those defined in commonly used dictionaries, should be interpreted as having a meaning that is consistent with their meaning in the context of the relevant art and will not be interpreted in an idealized or overly formal sense unless expressly so defined in the present specification.

Effects and features of the second and third aspects are to a large extent analogous to those described above in connection with the first aspect. Embodiments mentioned in relation to the first aspect are largely compatible with the second and third aspects.

The present disclosure will become apparent from the detailed description given below. The detailed description and specific examples disclose preferred embodiments of the disclosure by way of illustration only. Those skilled in the art understand from guidance in the detailed description that changes and modifications may be made within the scope of the disclosure. The description herein of any aspect or embodiment of the invention using terms such as “comprising”, “having,” “including,” or “containing” with reference to an element or elements is intended to provide support for a similar aspect or embodiment of the invention that “consists of”, “consists essentially of”, or “substantially comprises” that particular element or elements, unless otherwise stated or clearly contradicted by context, e.g. a composition described herein as comprising a particular element should be understood as also describing a composition consisting of that element, unless otherwise stated or clearly contradicted by context. It will be further understood that the terms “comprises,” “comprising,” “includes” and/or “including,” when used in this specification, specify the presence of stated features, integers, steps, operations, elements, and/or components, but do not preclude the presence or addition of one or more other features, integers, steps, operations, elements, components, and/or groups thereof.

As used herein, the singular forms “a,” “an,” and “the” are intended to include the plural forms, including “at least one,” unless the content clearly indicates otherwise. “At least one” is not to be construed as limiting “a” or “an.”

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The present invention relates to an absorbent insert ostomy device for insertion through an ostomy of a human being. The present invention further relates to various uses and methods of said device and uses of modified cellulose for various purposes.

In particular, the present invention relates to an absorbent insert ostomy device for insertion through an ostomy of a human being, the device being configured to absorb intestinal fluid and waste inside the intestines of the human being, the absorbent insert ostomy device comprising: an expandable elongated body extending along a longitudinal direction of the absorbent insert ostomy device and having a stoma end and an oral end spaced from the stoma end along the longitudinal direction, wherein the expandable elongated body comprises one or more absorbent materials; one or more withdrawal means attached to the stoma end of the expandable elongated body; and one or more compression means configured to maintain the expandable elongated body in a compressed state, having a first diameter along the longitudinal direction, prior to insertion of the absorbent insert ostomy device through the ostomy of the human being; wherein the expandable elongated body is configured to expand radially to a non-compressed state, having a second diameter along the longitudinal direction, when released from the one or more compression means upon insertion into the intestines of the human being through the ostomy; wherein the second diameter is larger than the first diameter; wherein the expandable elongated body comprising the absorbent material is further configured to be pulled out of the intestines of the human being by means of the one or more withdrawal means. Particularly disclosed in where the one or more absorbent materials is at least one or more cellulose derivatives, especially wherein the one or more absorbent materials at least comprises carboxymethyl cellulose.

Intestinal fluid may also be termed intestinal liquid and intestinal output.

The stoma end may also be termed the anal or distal end and the oral end may also be called the proximal end.

The absorbent material is preferably selected from one or more materials having a large absorbance capacity, i.e. the absorbency of the material is high. The material may further be selected from one or more materials which expands on absorption of liquids. By absorbency is meant absorbency assessed by any known method yielding absorbency in terms of weight per unit area, which preferably could be converted to absorbency in terms of weight ratio (g/g). Absorption could e.g. be measured by taking a sample measuring 5 cm times 5 cm and weighing it (denoted W1). The sample is then immersed in a physiological saline solution for 30 minutes at 37 degrees centigrade. The sample is then held to drip for 30 seconds before being weighed (denoted W2). Absorption (in g/g) is then equal to (W2−W1)/W1. Absorption (in g/cm²) is then equal to (W2−W1)/area.

Preferably, the absorbent insert ostomy device is held in a compressed state prior to insertion through the stoma of the subject, said compressed state ensuring that the insert device can be inserted through the stoma. The insert device, in the compressed state, may have a cylindrical shape, preferably slightly conical with a smaller diameter at one end. The insert device may be compressed both longitudinally and radially. As an example, the insert device may have a diameter of approximately 1 to 3 cm and a length of approximately 5 to 20 cm in the compressed state. In its non-compressed state, the insert device could preferably obtain a diameter corresponding to the body canal of the subject (in case of the ileum of a person the diameter could be 2 to 5 cm) and a length of approximately 20 to 40 cm. The absorbent material may therefore be configured such that the diameter perpendicular to the longitudinal axis increases by at least 100% from the compressed state to the non-compressed state. Further, the absorbent material may be configured such that the longitudinal axis (the length of the absorbent material) is increased by at least 100% from the compressed state to the non-compressed state. Additionally, the absorbent material may be configured to expand such that the overall volume of the absorbent material in the non-compressed state is at least factor 4, such as a factor 8, larger than the volume of the absorbent material in the compressed state.

The compression means configured to maintain the expandable elongated body in a compressed state may be realised in a number of different ways, e.g. by a coating and/or an insertion sleeve enclosing the absorbent material. The coating is preferably water-soluble and configured to dissolve upon exposure to the moisture, intestinal fluids, and/or heat levels inside the subject. Accordingly, the insert device may comprise a coating and/or an insertion sleeve enclosing the absorbent material. As an example, the absorbent material may be compressed and wrapped in a polymer film, such as a water-soluble synthetic polymer film, the polymer film thereby constituting the coating. An example of a suitable polymer film is a poly(vinyl alcohol) (PVA) film. However, other coatings may be used. Alternatively, the device is compressed in an applicator and inserted and released inside the body canal, e.g. the small intestine. The applicator material may be paper, cardboard or plastic. Another insertion method consists of an insertion probe in terms of a catheter-like thin tube that is placed inside the absorbent insert ostomy device and guides the absorbent insert ostomy device into place whereby the probe is pulled out. Another insertion method consists of a plastic sheet covering the ostomy device. When inserted, the plastic sheet is withdrawn from the ostomy device which is then in place in the intestine. Suited materials for a probe/guiding catheter are latex, silicone, rubber, plastic or nitinol. Upon placement of the absorbent insert ostomy device inside the body canal, e.g. ileum, of the subject, the insert device is configured to expand to a size once the water-soluble coating is dissolved and/or the insertion sleeve is removed. The insert device preferably expands both in the radial and longitudinal direction.

By non-compressed is meant the size when the one or more absorbent materials in in their fully wetted state, i.e. the second diameter being the diameter when the expandable elongated body is fully wetted.

The absorbent insert ostomy device comprises an expandable elongated body. The elongated body may comprise or be made entirely from an absorbent material. Alternatively, the elongated body may form a receptacle, such as a tube, filled or partly filled with the one or more absorbent materials. The absorbent materials may be provided as a woven fabric, a nonwoven fabric, or a foam. An example of a nonwoven fabric is an air-laid paper.

In one or more embodiments, the expandable elongated body expands on the absorption of intestinal fluid and/or intestinal waste. This could be assessed by simply substituting the intestinal waste with an isotonic brine solution.

In one or more embodiments, the one or more absorbent materials is a compressed body of cellulose which has been chemically modified to increase its absorbency to at least 19 g/g. This may be measured by a free swell method. The compressed body of (modified) cellulose will preferably expand on absorption of fluid.

In one or more embodiments, the ostomy device is a colostomy, an urostomy, an ileostomy, or a jejunostomy device.

An ileostomy is an opening in the belly (abdominal wall) that is made during surgery. It is usually needed because a problem is causing the ileum to not work properly, or a disease is affecting that part of the colon and it needs to be removed. The end of the ileum (the lowest part of the small intestine) is brought through this opening to form a stoma. An ileostomy may only be needed for a short time (temporary), maybe for 3 to 6 months, because that part of the colon needs time to rest and heal from a problem or disease. But sometimes a disease, such as cancer or inflammatory bowel disease, is more serious or chronic, and an ileostomy may be needed for the rest of a person's life (permanent). Besides usage in an ileostomy, the present invention can be used for other related purposes, such as preventing leakage from urostomies, colostomies, and jejunostomies. Sometimes jejunostomies and ileostomies are used interchangeably. Further envisioned may be the use of such device for the rectum, where it is used in connection with anal incontinence.

In one or more embodiments, the ostomy device is an ileostomy or a jejunostomy device.

In one or more embodiments, the ostomy device is for insertion through a colostomy, an urostomy, an ileostomy, or a jejunostomy.

In one or more embodiments, the ostomy device is for insertion through an ileostomy or a jejunostomy.

In one or more embodiments, the radial size of the expandable elongated body upon withdrawal near the ostomy, is substantially equal to the radial size of the expandable elongated body in the compressed state, prior to insertion of the absorbent insert ostomy device through the ostomy of the human being. In other words, the radial size of the expandable elongated body when removing the device preferably corresponds to the radial size in the compressed state. In this way there is certainty that the user can get the device out of the ostomy.

It is important that the device is formed in such a way, so that retention or encapsulation of the fluid occurs. This can also be obtained by choosing the right absorbent material, as e.g. device/elongated body will experience a slight pressure when it is to be extracted through the stoma hole.

In one or more embodiments, the expandable elongated body has a first radial width along the longitudinal direction when the expandable elongated body is in the compressed state, and wherein the expandable elongated body comprising the absorbent material is further configured to conform to a second radial width when the absorbent insert ostomy device is pulled out of the intestines of the human being by means of the one or more withdrawal means, wherein the second radial width is substantially equal to the first radial length.

In one or more embodiments, the expandable elongated body is rigid when the expandable elongated body is in the compressed state and flexible when the expandable elongated body is in the non-compressed state. In a preferred embodiment, upon insertion, the device is rigid, and preferably no longer than 10 cm in the longitudinal direction.

By rigid is meant that it is unable to bend or be forced out of shape when applying a slight pressure by the user's hand. Small bending at higher amount of pressure applied is acceptable, however, it should be such that during normal use it will not bend to any major extend. By flexible is meant that it is capable of bending easily without breaking when only applying very little force to the device, such as e.g. holding it by hand in a horizontal alignment (e.g. bending under the pressure of gravity).

In one or more embodiments, the expandable elongated body changes from being rigid to being flexible when the expandable elongated body expands radially from the compressed state to the non-compressed state.

In one or more embodiments, the one or more absorbent materials are selected from materials being biodegradable. A biodegradable material is a material, which can undergo biodegradation, which is the breakdown of organic matter by microorganisms, such as bacteria and fungi. In other words, if something is biodegradable, then, given the right conditions and presence of microorganisms, fungi, or bacteria, it will eventually break down to its basic components and blend back in with the earth. Ideally, but not always, these substances degrade without leaving any toxins behind.

In one or more embodiments, the one or more absorbent materials at least comprises modified cellulose, such as chemically modified cellulose.

The chemical modification may be carboxymethylation of cellulose, cotton, or a combination of the two and may be performed by contacting the material with a strong alkali, for example sodium hydroxide and a carboxymethylation agent such as chloroacetic acid or a salt thereof such as the sodium salt. These reagents may be applied to the material separately or together. The reaction is conveniently performed in an aqueous system which preferably comprises a water-miscible organic solvent such as ethanol or industrial methylated spirits in order to suppress swelling and dissolution of the carboxymethylated cellulose. A suitable method of conducting the modification is described in WO 00/01425. In a suitable method, the cellulosic body is loaded into a reaction vessel. Reaction fluid is pumped through the cellulosic material at 65 degrees centigrade for 90 minutes. The reaction fluid is a solution of an alkali (typically sodium hydroxide) and sodium monochloroacetate in industrial denatured alcohol. After the reaction time, the reaction is neutralised with acid and washed before being dried in a laboratory oven for 1 hour at 40 degrees centigrade.

In one or more embodiments, the one or more absorbent materials at least consists of a plurality of monomer units linked to each other via ether bonds, ester bonds, amide bonds, or combinations hereof, wherein said monomer units are selected from molecules having formula X:

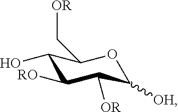

wherein R can be H, CH₂COONa, CH₂COOK, or CH₂COOH.

In one or more embodiments, the one or more absorbent materials at least comprises a cellulose derivative selected from methyl cellulose (MC), hydroxypropyl cellulose (HPC), hydroxypropylmethyl cellulose (HPMC), ethyl cellulose (EC), sodium carboxymethyl cellulose (NaCMC), carboxymethyl cellulose (CMC), or combinations thereof.

In one or more embodiments, the one or more absorbent materials are in the form of a salt, such as a sodium or a potassium salt. A salt herein is a chemical compound consisting of an ionic assembly of positively charged cations and negatively charged anions, which results in a compound with no net electric charge. This can be a component ion such as the carboxylate (RCOO⁻) together with e.g. sodium (Na⁺).

In one or more embodiments, the one or more absorbent materials originate from one or more cellulose sources, such as one or more natural cellulose sources.

In one or more embodiments, the one or more absorbent materials are selected from carboxymethyl cellulose (CMC), alginate, super absorbent polymers (SAP), super absorbent fibres, plant fibres/polymers, or combinations thereof.

In one or more embodiments, the one or more absorbent materials at least comprises carboxymethyl cellulose.

In one or more embodiments, the one or more absorbent materials at least comprises carboxymethyl cellulose fibres having a degree of substitution of at least 0.1, such as a degree of substitution of at least 0.2, or such as a degree of substitution of at least 0.3. The degree of substitution (DS) of a polymer is the (average) number of substituent groups attached per base unit (in the case of condensation polymers) or per monomeric unit (in the case of addition polymers). The term has been mainly used in cellulose chemistry.

Sodium carboxymethylcellulose (CMC for short) is a kind of high-polymer cellulose ether made from natural cellulose by chemical modification. CMC thickener is a widely used and the most cost-effective thickening agent. As can be seen from the structure formula, there are three hydroxyl groups in each glucose unit, namely C2, C3, and C6 hydroxyl groups. The number of hydrogens in hydroxyl group of glucose unit replaced by carboxymethyl can be expressed by degree of substitution (DS). If all the three hydroxyl groups in each glucose unit are replaced by carboxymethyl, the degree of substitution can be defined as 3. The degree of substitute has a direct impact on the solubility, emulsibility, thickening property, stability, acid resistance and salt tolerance.

It is generally thought that Sodium carboxymethyl cellulose has better emulsifying property when the degree of substitution is 0.6-0.7. Furthermore, with the increase of DS, other properties can be improved accordingly. When the degree of substitution is over 0.8, the resistance to acids and salts can be remarkably enhanced. To high degree of substitution also makes the polymer soluble in e.g. water; hence, in one or more embodiments, the one or more absorbent materials at least comprises carboxymethyl cellulose fibres having a degree of substitution of no more than 0.8, such as a degree of substitution of no more than 0.7, or such as a degree of substitution of no more than 0.6.

In one or more embodiments, the one or more absorbent materials comprises at least 50% by weight of carboxymethyl cellulose compared to the total weight of the expandable elongated body, such as at least 60% by weight, such as at least 70% by weight, such as at least 80% by weight, or such as at least 90% by weight of carboxymethyl cellulose compared to the total weight of the expandable elongated body.

In one or more embodiments, the one or more absorbent materials is carboxymethyl cellulose.

In one or more embodiments, the carboxymethyl cellulose is in the form of a powder, fibre, non-woven, gel, or combinations thereof.

In one or more embodiments, the carboxymethyl cellulose is in the form of rope shaped, sheet shaped, or combinations thereof.

In one or more embodiments, the carboxymethyl cellulose is in the form of sheets.

In one or more embodiments, the carboxymethyl cellulose is in the form of sodium carboxymethylcellulose fibres.

In one or more embodiments, the carboxymethyl cellulose fibres has a mass per unit area from 120 g/m² to 180 g/m², such as 130 g/m² to 170 g/m², such as 140 g/m² to 160 g/m², or such as 145 g/m² to 155 g/m². The mass per unit of the CMC material is measured according to ISO 9073-1:1989.

In one or more embodiments, the carboxymethyl cellulose fibres has a liquid absorption of at least 15 g per 100 cm², such as a liquid absorption of at least 20 g/100 cm². The liquid absorption is measured according to EN 13726-1:2002.

In one or more embodiments, the carboxymethyl cellulose fibres a tensile strength in its dry state (i.e. prior to wetting) of at least 3 Newton per centimetre, such as at least 5 N/cm, such as at least 10 N/cm, or such as at least 12 N/cm.

In one or more embodiments, the carboxymethyl cellulose fibres a tensile strength in its wet state (i.e. after wetting) of at least 0.2 Newton per centimetre, such as at least 0.3 N/cm, such as at least 0.4 N/cm, such as at least 0.5 N/cm, or such as at least 0.6 N/cm. The tensile strength (dry or wet) is both measured according to ISO 13934-1:2013.

In one or more embodiments, the carboxymethyl cellulose fibres has a loss on drying of no more than 25%, such as no more than 20%, or such as no more than 18%. The loss on drying is measured according to JYWN-QM-P02-SOP162.

In one or more embodiments, the one or more absorbent materials, such as carboxymethyl cellulose, has a heavy metal content of no more than 20 microgram per gram. The heavy metal content is measured according to JYWN-QM-P02-SOP162.

In one or more embodiments, the one or more absorbent materials further comprises alginate, cellulose, cotton, chitosan, or combination thereof.

In one or more embodiments, the expandable elongated body further comprises one or more antibacterial compounds, such as silver particles.

In one or more embodiments, the expandable elongated body further comprises one or more reinforcement materials.

In one or more embodiments, the one or more reinforcement materials are selected from non-woven fibres, woven fibres, or combinations thereof.

In one or more embodiments, the one or more reinforcement materials are selected from nylon, polypropylene, cotton, cellulose material combined with fibres of polymer, or combinations thereof.

In one or more embodiments, the one or more absorbent materials are selected from material having an intestinal fluid and waste absorbing capacity of at least 500%, such as at least 1000%, such as at least 2000%, or such as at least 3000%. These data could e.g. be obtained as described above, by weighing the material before and after absorption of intestinal fluids (or simply just isotonic water solution).

In one or more embodiments, the one or more absorbent materials are selected from material having a water absorbance capacity of at least a factor 5, such as a factor 10, such as at least a factor 20, or such as at least a factor 30.

In one or more embodiments, the first diameter is equal to or less than the diameter of the ostomy of the human being.

In one or more embodiments, the second diameter is equal to or larger than the diameter of the intestines of the human being.

In one or more embodiments, the expandable elongated body is configured to be further compressed along the longitudinal direction by the ostomy upon insertion of the absorbent insert ostomy device through the ostomy of the human being.

In one or more embodiments, the second diameter is larger than the first diameter by at least a factor 1.25, such as by at least a factor 1.5, such as by at least a factor 1.75, such as by at least a factor 2, or such as by at least a factor 2.5.

In one or more embodiments, the ratio of the thickness of the expandable elongated body along the longitudinal direction pre-hydration to the thickness of the expandable elongated body along the longitudinal direction post hydration is greater than 1.25. The pre/post hydration thickness may be measured by a method described herein or by any known method to measure such.

In one or more embodiments, the ratio of the thickness of the expandable elongated body along the longitudinal direction pre-hydration to the thickness of the expandable elongated body along the longitudinal direction post hydration is greater than 1.5, such as 1.75, such as 2, or such as greater than 2.5.

In one or more embodiments, the ratio of the thickness of the one or more absorbent materials along the longitudinal direction of the expandable elongated body pre-hydration to the thickness of the one or more absorbent materials along the longitudinal direction of the expandable elongated body post hydration is greater than 1.25. The pre/post hydration thickness may be measured by a method described herein or by any known method to measure such.

In one or more embodiments, the ratio of the thickness of the one or more absorbent materials along the longitudinal direction of the expandable elongated body pre-hydration to the thickness of the one or more absorbent materials along the longitudinal direction of the expandable elongated body post hydration is greater than 1.5, such as 1.75, such as 2, or such as greater than 2.5.

In one or more embodiments, the first diameter is 1.75 cm or less, such as 1.50 cm or less, or such as 1.25 cm or less.

In one or more embodiments, the first diameter is between 1.00 and 1.75 cm, such as between 1.00 and 1.50 cm, or such as between 1.00 and 1.25 cm.

In one or more embodiments, the second diameter is at least 2 cm, such as at least 2.5 cm, or such as at least 3 cm.

In one or more embodiments, the second diameter is between 2 and 5 cm, such as between 2 and 4 cm, such as between 2 and 3 cm, or such as between 2.5 and 4 cm.

In one or more embodiments, the length of the expandable elongated body in the compressed state along the longitudinal direction is 20 cm or less, such as 17.5 cm or less, such as 15 cm or less, such as 12.5 cm or less, or such 10 cm or less.

In one or more embodiments, the length of the expandable elongated body in the non-compressed state along the longitudinal direction is at least 12.5 cm, such as at least 15 cm, such as at least 20 cm, such as at least 25 cm, such as at least 30 cm, such as at least 35 cm, or such as at least 40 cm.

In one or more embodiments, the expandable elongated body in the non-compressed state has a second diameter of at least 2 cm, such as from 2 to 5 cm, and length along the longitudinal direction of at least 20 cm, such as from 20 to 40 cm.

High absorption capacity may be needed from the device. It could be important that the device is able to absorb at least between 100 mL and 200 mL to provide value for the users, however, it may still be significantly compressed when being inserted (e.g. such as dimension of about 10×1.5 cm) to be able to enter through the stoma hole without too much discomfort for the user. Because of this, it is preferred that the material has a high absorbency.

In one or more embodiments, the absorbent insert ostomy device has an absorption capacity of at least 50 millilitres, such as at least 75 millilitres, such as at least 100 millilitres, such as at least 125 millilitres, such as at least 150 millilitres, such as at least 175 millilitres, such as at least 200 millilitres, or such as at least 250 millilitres.

In one or more embodiments, the one or more absorbent materials has an absorption capacity of at least 50 millilitres, such as at least 75 millilitres, such as at least 100 millilitres, such as at least 125 millilitres, such as at least 150 millilitres, such as at least 175 millilitres, such as at least 200 millilitres, or such as at least 250 millilitres.

In one or more embodiments, the expandable elongated body is curled around the longitudinal axis to form a helix comprising a plurality of turns.

In one or more embodiments, the expandable elongated body comprises a plurality of flexible protrusions extending radially from the body, such that the elongated body resembles a brush.

In one or more embodiments, the expandable elongated body is curled around the longitudinal axis to form a helix comprising a plurality of turns and further comprises a plurality of flexible protrusions extending radially from the body.

In one or more embodiments, the expandable elongated body is curled around the longitudinal axis to form a helix comprising a plurality of turns and further comprises a plurality of flexible protrusions extending radially from the body, wherein the plurality of flexible protrusions has a hair-like configuration.

In one or more embodiments, the elongated body is provided as a flexible tube, which is partly or entirely filled with the one or more absorbent materials. In one or more embodiments, the flexible tube is a woven crinoline tube comprising a material selected from the group of: nylon, polyester, polyvinylidene fluoride (PVDF), polypropylene, or combinations thereof.

In one or more embodiments, the expandable elongated body is formed from a sheet, which is rolled into an elongated conical shape.

In one or more embodiments, the expandable elongated body is formed from a tube having a closed end, said tube being folded one or more times inside itself, wherein the tube is configured to unfold upon pulling the device out of the ostomy by use of the withdrawal means.

In one or more embodiments, the expandable elongated body comprises a plurality of cylinders, each cylinder comprising an absorbent material, and wherein withdrawal means is attached to each cylinder, such that the plurality of cylinders may be withdrawn from the subject individually.

In one or more embodiments, the expandable elongated body comprises a support structure configured to increase the longitudinal axial stability of the body, wherein said support structure comprises a material selected from the group of: silicone, rubber, latex, polyethylene, polyvinyl chloride, nitinol, or combinations thereof.

In one or more embodiments, the absorbent insert ostomy is to collect intestinal fluid and waste from the intestines of a subject.

In one or more embodiments, the absorbent insert ostomy device is for insertion through an ostomy of a human being.

In one or more embodiments, the absorbent insert ostomy device is an absorbent insert ostomy device.

In one or more embodiments, the modified cellulose is biodegradable.

In one or more embodiments, the modified cellulose at least comprises chemically modified cellulose.

In one or more embodiments, the modified cellulose consists of a plurality of monomer units linked to each other via ether bonds, ester bonds, amide bonds, or combinations hereof, wherein said monomer units are selected from molecules having formula X:

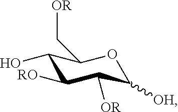

wherein R can be H, CH₂COONa, CH₂COOK, or CH₂COOH.

In one or more embodiments, the modified cellulose at least comprises a cellulose derivative selected from methyl cellulose (MC), hydroxypropyl cellulose (HPC), hydroxypropylmethyl cellulose (HPMC), ethyl cellulose (EC), sodium carboxymethyl cellulose (NaCMC), carboxymethyl cellulose (CMC), or combinations thereof.

In one or more embodiments, the modified cellulose is in the form of a salt, such as a sodium or a potassium salt.

In one or more embodiments, the modified cellulose originates from one or more cellulose sources, such as one or more natural cellulose sources.

In one or more embodiments, the modified cellulose at least comprises carboxymethyl cellulose.

In one or more embodiments, the modified cellulose at least comprises carboxymethyl cellulose fibres having a degree of substitution of at least 0.1, such as a degree of substitution of at least 0.2, or such as a degree of substitution of at least 0.3.

In one or more embodiments, the modified cellulose at least comprises carboxymethyl cellulose fibres having a degree of substitution of no more than 0.8, such as a degree of substitution of no more than 0.7, or such as a degree of substitution of no more than 0.6.

In one or more embodiments, the modified cellulose at least comprises 50% by weight of carboxymethyl cellulose, such as at least 60% by weight, such as at least 70% by weight, such as at least 80% by weight, or such as at least 90% by weight of carboxymethyl cellulose.

In one or more embodiments, the carboxymethyl cellulose is in the form of a powder, fibre, non-woven, gel, or combinations thereof.

In one or more embodiments, the carboxymethyl cellulose is in the form of rope shaped, sheet shaped, or combinations thereof.

In one or more embodiments, the carboxymethyl cellulose is in the form of sheets.

The absorbent insert ostomy device as disclosed herein may be produced using various methods. In one or more embodiments, two identical bands of absorbent material, such as CMC, are placed on top of each other. In a preferred embodiment, the length of each is from 50 cm to 100 cm, and the width of each is from 2.5 cm to 4.0 cm.

In one or more embodiments, parallel cuts are made in the whole length of the identical bands, from both sides perpendicular to the longitudinal direction, leaving a central spine that holds the identical bands together and creates a hair-like structure in the identical bands.

In one or more embodiments, the two identical bands are then sewn together along the central spine with an elastic band that is stretched to 80-100% of the elastic bads maximal stretching capacity during the sewing process. In a preferred embodiment, the elastic band is attached to the full length of the identical bands and continues a further 10 cm to 30 cm from where the bands end, leaving a solitary elastic band string. Such string may be used as a withdrawal string, i.e. the one or more withdrawal means. Upon relaxation of the elastic band, the two identical bands, now sewed to the elastic band, will curl up into a spiral-shaped device of approximately 20 cm to 35 cm in length (depending on the initial length of the identical bands) with an appendix of approximately 10 cm to 30 cm of the elastic band without absorbent material.

In one or more embodiments, the absorbent insert ostomy device is then compressed into a length of 3 cm to 8 cm and a diameter of 0.5 cm to 1.8 cm, onto which a poly(vinyl alcohol) (PVA) film is wrapped around for permanently fixating the device in the compressed state.

In one or more embodiments, the device in the compressed state is inserted into the stoma, whereby the secretion of the stoma causes the PVA film to dissolve. The device will then slowly expand in length and width as it absorbs the liquid faeces inside the intestines, hereby enabling a leak-tight (or semi-leak-tight) closure, by having a diameter of approximately 3 cm which is bigger than a typical stoma opening of 1.5 cm. The device is kept in place by the abdominal wall as the peristaltic movements pushes it towards the abdominal wall.

In one or more embodiments, the hair-like structure is configured to collect solid parts of food in the faeces, which can then be withdrawn from the intestine together with the liquid faeces, when removing the device from the stoma. Further, the hair-like structure may ensure a large surface area of the absorbing material hereby ensuring a large absorption capacity.

In one or more embodiments, the size of the absorbent insert ostomy device is designed to fit the anatomical dimensions of a human intestine.

When describing the embodiments, the combinations and permutations of all possible embodiments have not been explicitly described. Nevertheless, the mere fact that certain measures are recited in mutually different dependent claims or described in different embodiments does not indicate that a combination of these measures cannot be used to advantage. The present invention envisages all possible combinations and permutations of the described embodiments.

All headings and sub-headings are used herein for convenience only and should not be construed as limiting the invention in any way.
1. An absorbent insert ostomy device for insertion through an ostomy of a human being, the device being configured to absorb intestinal fluid and waste inside the intestines of the human being, the absorbent insert ostomy device comprising:
   an expandable elongated body extending along a longitudinal direction of the absorbent insert ostomy device and having a stoma end and an oral end spaced from the stoma end along the longitudinal direction, wherein the expandable elongated body comprises one or more absorbent materials;
   one or more withdrawal means attached to the stoma end of the expandable elongated body; and
   one or more compression means configured to maintain the expandable elongated body in a compressed state, having a first diameter along the longitudinal direction, prior to insertion of the absorbent insert ostomy device into the ostomy of the human being;
wherein the expandable elongated body is configured to expand radially to a non-compressed state, having a second diameter along the longitudinal direction, when released from the one or more compression means upon insertion into the intestines of the human being through the ostomy;
wherein the second diameter is larger than the first diameter; and
wherein the expandable elongated body comprising the absorbent material is further configured to be pulled out of the intestines of the human being by means of the one or more withdrawal means.
2. The absorbent insert ostomy device according to item 1, wherein the ostomy device is a colostomy, an urostomy, an ileostomy, or a jejunostomy device.
3. The absorbent insert ostomy device according to any of the preceding items, wherein the ostomy device is an ileostomy or a jejunostomy device.
4. The absorbent insert ostomy device according to any of the preceding items, wherein the ostomy device is for insertion through a colostomy, an urostomy, an ileostomy, or a jejunostomy.
5. The absorbent insert ostomy device according to any of the preceding items, wherein the ostomy device is for insertion through an ileostomy or a jejunostomy.
6. The absorbent insert ostomy device according to any of the preceding items, wherein the radial size of the expandable elongated body upon withdrawal near the ostomy, is substantially equal to the radial size of the expandable elongated body in the compressed state, prior to insertion of the absorbent insert ostomy device through the ostomy of the human being.
7. The absorbent insert ostomy device according to any of the preceding items, wherein the expandable elongated body has a first radial width along the longitudinal direction when the expandable elongated body is in the compressed state, and wherein the expandable elongated body comprising the absorbent material is further configured to conform to a second radial width when the absorbent insert ostomy device is pulled out of the intestines of the human being by means of the one or more withdrawal means, wherein the second radial width is substantially equal to the first radial length.
8. The absorbent insert ostomy device according to any of the preceding items, wherein the expandable elongated body is rigid when the expandable elongated body is in the compressed state and flexible when the expandable elongated body is in the non-compressed state.
9. The absorbent insert ostomy device according to any of the preceding items, wherein the expandable elongated body changes from being rigid to being flexible when the expandable elongated body expands radially from the compressed state to the non-compressed state.
10. The absorbent insert ostomy device according to any of the preceding items, wherein the one or more absorbent materials are selected from materials being biodegradable.
11. The absorbent insert ostomy device according to any of the preceding items, wherein the one or more absorbent materials at least comprises modified cellulose, such as chemically modified cellulose.
12. The absorbent insert ostomy device according to any of the preceding items, wherein the one or more absorbent materials at least consists of a plurality of monomer units linked to each other via ether bonds, ester bonds, amide bonds, or combinations hereof, wherein said monomer units are selected from molecules having formula X:

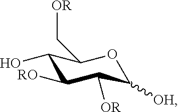

wherein R can be H, CH₂COONa, CH₂COOK, or CH₂COOH.
13. The absorbent insert ostomy device according to any of the preceding items, wherein the one or more absorbent materials at least comprises a cellulose derivative selected from methyl cellulose (MC), hydroxypropyl cellulose (HPC), hydroxypropylmethyl cellulose (HPMC), ethyl cellulose (EC), sodium carboxymethyl cellulose (NaCMC), carboxymethyl cellulose (CMC), or combinations thereof.
14. The absorbent insert ostomy device according to any of the preceding items, wherein the one or more absorbent materials are in the form of a salt, such as a sodium or a potassium salt.
15. The absorbent insert ostomy device according to any of the preceding items, wherein the one or more absorbent materials originate from one or more cellulose sources, such as one or more natural cellulose sources.
16. The absorbent insert ostomy device according to any of the preceding items, wherein the one or more absorbent materials are selected from carboxymethyl cellulose (CMC), alginate, super absorbent polymers (SAP), super absorbent fibers, plant fibers/polymers, or combinations thereof.
17. The absorbent insert ostomy device according to any of the preceding items, wherein the one or more absorbent materials at least comprises carboxymethyl cellulose.
18. The absorbent insert ostomy device according to any of the preceding items, wherein the one or more absorbent materials at least comprises carboxymethyl cellulose fibres having a degree of substitution of at least 0.1, such as a degree of substitution of at least 0.2, or such as a degree of substitution of at least 0.3.
19. The absorbent insert ostomy device according to any of the preceding items, wherein the one or more absorbent materials at least comprises carboxymethyl cellulose fibres having a degree of substitution of no more than 0.8, such as a degree of substitution of no more than 0.7, or such as a degree of substitution of no more than 0.6.
20. The absorbent insert ostomy device according to any of the preceding items, wherein the one or more absorbent materials comprises at least 50% by weight of carboxymethyl cellulose compared to the total weight of the expandable elongated body, such as at least 60% by weight, such as at least 70% by weight, such as at least 80% by weight, or such as at least 90% by weight of carboxymethyl cellulose compared to the total weight of the expandable elongated body.
21. The absorbent insert ostomy device according to any of the preceding items, wherein the one or more absorbent materials is carboxymethyl cellulose.
22. The absorbent insert ostomy device according to any of the preceding items, wherein the carboxymethyl cellulose is in the form of a powder, fibre, non-woven, gel, or combinations thereof.
23. The absorbent insert ostomy device according to any of the preceding items, wherein the carboxymethyl cellulose is in the form of rope shaped, sheet shaped, or combinations thereof.
24. The absorbent insert ostomy device according to any of the preceding items, wherein the carboxymethyl cellulose is in the form of sheets.
25. The absorbent insert ostomy device according to any of the preceding items, wherein the one or more absorbent materials further comprises alginate, cellulose, cotton, chitosan, or combination thereof.
26. The absorbent insert ostomy device according to any of the preceding items, wherein the expandable elongated body further comprises one or more antibacterial compounds, such as silver particles.
27. The absorbent insert ostomy device according to any of the preceding items, wherein the expandable elongated body further comprises one or more reinforcement materials.
28. The absorbent insert ostomy device according to item 27, wherein the one or more reinforcement materials are selected from non-woven fibres, woven fibres, or combinations thereof.
29. The absorbent insert ostomy device according to any one of items 27-28, wherein the one or more reinforcement materials are selected from nylon, polypropylene, cotton, cellulose material combined with fibres of polymer, or combinations thereof.
30. The absorbent insert ostomy device according to any of the preceding items, wherein the one or more absorbent materials are selected from material having an intestinal fluid and waste absorbing capacity of at least 500%, such as at least 1000%, such as at least 2000%, or such as at least 3000%.
31. The absorbent insert ostomy device according to any of the preceding items, wherein the one or more absorbent materials are selected from material having a water absorbance capacity of at least a factor 5, such as a factor 10, such as at least a factor 20, or such as at least a factor 30.
32. The absorbent insert ostomy device according to any of the preceding items, wherein the first diameter is equal to or less than the diameter of the ostomy of the human being.
33. The absorbent insert ostomy device according to any of the preceding items, wherein the second diameter is equal to or larger than the diameter of the intestines of the human being.
34. The absorbent insert ostomy device according to any of the preceding items, wherein the expandable elongated body is configured to be further compressed along the longitudinal direction by the ostomy upon insertion of the absorbent insert ostomy device through the ostomy of the human being.
35. The absorbent insert ostomy device according to any of the preceding items wherein the second diameter is larger than the first diameter by at least a factor 1.25, such as by at least a factor 1.5, such as by at least a factor 1.75, such as by at least a factor 2, or such as by at least a factor 2.5.
36. The absorbent insert ostomy device according to any of the preceding items, wherein the first diameter is 1.75 cm or less, such as 1.50 cm or less, or such as 1.25 cm or less.
37. The absorbent insert ostomy device according to any of the preceding items, wherein the first diameter is between 1.00 and 1.75 cm, such as between 1.00 and 1.50 cm, or such as between 1.00 and 1.25 cm.
38. The absorbent insert ostomy device according to any of the preceding items, wherein the second diameter is at least 2 cm, such as at least 2.5 cm, or such as at least 3 cm.
39. The absorbent insert ostomy device according to any of the preceding items, wherein the second diameter is between 2 and 5 cm, such as between 2 and 4 cm, such as between 2 and 3 cm, or such as between 2.5 and 4 cm.
40. The absorbent insert ostomy device according to any of the preceding items, wherein the length of the expandable elongated body in the compressed state along the longitudinal direction is 20 cm or less, such as 17.5 cm or less, such as 15 cm or less, such as 12.5 cm or less, or such as 10 cm or less.
41. The absorbent insert ostomy device according to any of the preceding items, wherein the length of the expandable elongated body in the non-compressed state along the longitudinal direction is at least 12.5 cm, such as at least 15 cm, such as at least 20 cm, such as at least 25 cm, such as at least 30 cm, such as at least 35 cm, or such as at least 40 cm.
42. The absorbent insert ostomy device according to any of the preceding items, wherein the expandable elongated body in the non-compressed state has a second diameter of at least 2 cm, such as from 2 to 5 cm, and a length along the longitudinal direction of at least 20 cm, such as from 20 to 40 cm.
43. The absorbent insert ostomy device according to any of the preceding items, wherein the absorbent insert ostomy device has an absorption capacity of at least 50 millilitres, such as at least 75 millilitres, such as at least 100 millilitres, such as at least 125 millilitres, such as at least 150 millilitres, such as at least 175 millilitres, such as at least 200 millilitres, or such as at least 250 millilitres.
44. The absorbent insert ostomy device according to any of the preceding items, wherein the one or more absorbent materials has an absorption capacity of at least 50 millilitres, such as at least 75 millilitres, such as at least 100 millilitres, such as at least 125 millilitres, such as at least 150 millilitres, such as at least 175 millilitres, such as at least 200 millilitres, or such as at least 250 millilitres.
45. The absorbent insert ostomy device according to any one of items 1-44, wherein the expandable elongated body is curled around the longitudinal axis to form a helix comprising a plurality of turns.
46. The absorbent insert ostomy device according to any one of items 1-44, wherein the expandable elongated body comprises a plurality of flexible protrusions extending radially from the body, such that the elongated body resembles a brush.
47. The absorbent insert ostomy device according to any one of items 1-44, wherein the elongated body is provided as a flexible tube, which is partly or entirely filled with the one or more absorbent materials.
48. The absorbent insert ostomy device according to item 47, wherein the flexible tube is a woven crinoline tube comprising a material selected from the group of: nylon, polyester, polyvinylidene fluoride (PVDF), polypropylene, or combinations thereof.
49. The absorbent insert ostomy device according to any one of items 1-44, wherein the expandable elongated body is formed from a sheet, which is rolled into an elongated conical shape.
50. The absorbent insert ostomy device according to any one of items 1-44, wherein the expandable elongated body is formed from a tube having a closed end, said tube being folded one or more times inside itself, wherein the tube is configured to unfold upon pulling the device out of the ostomy by use of the withdrawal means.
51. The absorbent insert ostomy device according to any one of items 1-44, wherein the expandable elongated body comprises a plurality of cylinders, each cylinder comprising an absorbent material, and wherein withdrawal means is attached to each cylinder, such that the plurality of cylinders may be withdrawn from the subject individually.
52. The absorbent insert ostomy device according to any of the preceding items, wherein the expandable elongated body comprises a support structure configured to increase the longitudinal axial stability of the body, wherein said support structure comprises a material selected from the groups of silicone, rubber, latex, polyethylene, polyvinyl chloride, nitinol, or combinations thereof.
53. Use of an absorbent insert ostomy device according to any one of items 1-52 to collect intestinal fluid and waste from the intestines of a subject.
54. Use of an absorbent insert ostomy device according to any one of items 1-52 for insertion through an ostomy of a human being.
55. A method for collecting intestinal waste from the intestines of a subject, the method comprising the steps of:
   inserting the absorbent insert ostomy device according to any one of items 1-52 through the ostomy of a subject, hereby placing the device inside the intestines of the subject;
   letting the absorbent material absorb intestinal waste from the subject; and withdrawing the absorbent insert ostomy device from the subject by use of the one or more withdrawal means.
56. Use of modified cellulose for producing an absorbent insert ostomy device according to any one of items 1-52.
57. Use of modified cellulose as an absorbent material in an absorbent insert ostomy device.
58. Use of modified cellulose as an absorbent material in an absorbent insert ostomy device according to item 57, wherein the absorbent insert ostomy device is an absorbent insert ostomy device according to any one of items 1-52.
59. Use of modified cellulose for absorbing intestinal fluid and waste inside the intestines of the human being.
60. Use of modified cellulose according to any one of items 56-59, wherein the modified cellulose is biodegradable.
61. Use of modified cellulose according to any one of items 56-60, wherein the modified cellulose at least comprises chemically modified cellulose.
62. Use of modified cellulose according to any one of items 56-61, wherein the modified cellulose consists of a plurality of monomer units linked to each other via ether bonds, ester bonds, amide bonds, or combinations hereof, wherein said monomer units are selected from molecules having formula X:

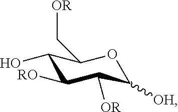

wherein R can be H, CH₂COONa, CH₂COOK, or CH₂COOH.
63. Use of modified cellulose according to any one of items 56-62, wherein the modified cellulose at least comprises a cellulose derivative selected from methyl cellulose (MC), hydroxypropyl cellulose (HPC), hydroxypropylmethyl cellulose (HPMC), ethyl cellulose (EC), sodium carboxymethyl cellulose (NaCMC), carboxymethyl cellulose (CMC), or combinations thereof.
64. Use of modified cellulose according to any one of items 56-63, wherein the modified cellulose is in the form of a salt, such as a sodium or a potassium salt.
65. Use of modified cellulose according to any one of items 56-64, wherein the modified cellulose originates from one or more cellulose sources, such as one or more natural cellulose sources.
66. Use of modified cellulose according to any one of items 56-65, wherein the modified cellulose at least comprises carboxymethyl cellulose.
67. Use of modified cellulose according to any one of items 56-66, wherein the modified cellulose at least comprises carboxymethyl cellulose fibres having a degree of substitution of at least 0.1, such as a degree of substitution of at least 0.2, or such as a degree of substitution of at least 0.3.
68. Use of modified cellulose according to any one of items 56-67, wherein the modified cellulose at least comprises carboxymethyl cellulose fibres having a degree of substitution of no more than 0.8, such as a degree of substitution of no more than 0.7, or such as a degree of substitution of no more than 0.6.
69. Use of modified cellulose according to any one of items 56-68, wherein the modified cellulose at least comprises 50% by weight of carboxymethyl cellulose, such as at least 60% by weight, such as at least 70% by weight, such as at least 80% by weight, or such as at least 90% by weight of carboxymethyl cellulose.
70. Use of modified cellulose according to any one of items 56-69, wherein the carboxymethyl cellulose is in the form of a powder, fibre, non-woven, gel, or combinations thereof.
71. Use of modified cellulose according to any one of items 56-70, wherein the carboxymethyl cellulose is in the form of rope shaped, sheet shaped, or combinations thereof.
72. Use of modified cellulose according to any one of items 56-71, wherein the carboxymethyl cellulose is in the form of sheets.
73. An absorbent insert ostomy device for insertion through an ostomy of a human being, the device being configured to absorb intestinal fluid and waste inside the intestines of the human being and to form a leak-tight closure of the ostomy, the absorbent insert ostomy device comprising:
   an expandable elongated body extending along a first longitudinal axis and having a stoma end and an oral end spaced from the stoma end along the first longitudinal axis, wherein the expandable elongated body comprises one or more absorbent materials;
   one or more withdrawal means attached to the stoma end of the expandable elongated body; and
   one or more compression means configured to maintain the expandable elongated body in a compressed state, having a first diameter along the first longitudinal axis, prior to insertion of the absorbent insert ostomy device through the ostomy of the human being;
wherein the expandable elongated body is configured to expand radially to a non-compressed state, having a second diameter along the first longitudinal axis, when released from the one or more compression means upon insertion into the intestines of the human being through the ostomy;
wherein the second diameter is larger than the first diameter, the first diameter being equal to or less than the diameter of the ostomy of the human being and the second diameter being equal to or larger than the diameter of the intestines of the human being;
wherein the expandable elongated body comprising the absorbent material is further configured to conform to a third diameter along a second axis when the absorbent insert ostomy device is pulled out of the intestines of the human being by means of the one or more withdrawal means;
wherein the second axis is an axis extending from the one or more withdrawal means through the ostomy, the second axis being perpendicular to a transverse plane of the ostomy; and
wherein the third diameter is substantially equal to the diameter of the ostomy.

The present disclosure will now be described with reference to the accompanying drawings, in which preferred example embodiments of the disclosure are shown. The disclosure may, however, be embodied in other forms and should not be construed as limited to the herein disclosed embodiments. The disclosed embodiments are provided to fully convey the scope of the disclosure to the skilled person.

Various examples are described hereinafter with reference to the figures. It should also be noted that the figures are only intended to facilitate the description of the examples. They are not intended as an exhaustive description of the claimed invention or as a limitation on the scope of the claimed invention. In addition, an illustrated example needs not have all the aspects or advantages shown. An aspect or an advantage described in conjunction with a particular example is not necessarily limited to that example and can be practiced in any other examples even if not so illustrated, or if not so explicitly described.

### DETAILED DESCRIPTION OF THE DRAWINGS

The above objects, as well as additional objects, features and advantages of the present disclosure will be more fully appreciated by reference to the following illustrative and non-limiting detailed description of example embodiments of the present disclosure, when taken in conjunction with the accompanying drawings.

FIG. 1A shows the absorbent insert ostomy device according to one embodiment of the present disclosure. This figure illustrates the insert device when it is in a compressed state and when it is placed in the body canal of a subject. This embodiment of the insert device comprises an elongated body, which is curled around a longitudinal axis of the body to form a helix comprising a plurality of turns.

FIG. 1B shows the absorbent insert ostomy device according to another embodiment of the present disclosure. This figure illustrates the insert device when it is in a compressed state and when it is placed in the body canal of a subject. This embodiment of the insert device comprises an elongated body with a plurality of flexible protrusions extending radially from the body, such that the elongated body resembles a brush.

FIG. 1C shows the absorbent insert ostomy device according to another embodiment of the present disclosure. This figure illustrates the insert device when it is in a compressed state and when it is placed in the body canal of a subject. In this embodiment, the elongated body of the insert device is provided as a flexible tube, which is preferably partly or entirely filled with the absorbent material.

FIG. 1D shows the absorbent insert ostomy device according to another embodiment of the present disclosure. This figure illustrates the insert device when it is in a compressed state and when it is placed in the body canal of a subject. In this embodiment, the elongated body of the insert device is formed from a sheet, which is rolled into an elongated conical shape.

FIG. 1E shows the absorbent insert ostomy device according to another embodiment of the present disclosure. This figure illustrates the insert device when it is in a compressed state and when it is placed in the body canal of a subject. In this embodiment, the elongated body of the insert device is formed from a tube having a closed end, said tube being folded one or more times inside itself, wherein the tube is configured to unfold upon pulling the device out of the stoma by use of the withdrawal means.

FIG. 1F shows the absorbent insert ostomy device according to another embodiment of the present disclosure. This figure illustrates the insert device when it is in a compressed state and when it is placed in the body canal of a subject. In this embodiment, the elongated body of the insert device is formed from a plurality of cylinders, each cylinder comprising an absorbent material.

FIGS. 2A-2F shows a non-compressed state of the insert device according to the corresponding (compressed) embodiment of FIG. 1A-1F. Hence, FIG. 2A is the non-compressed (expanded) state of the embodiment shown in FIG. 1A and so forth. For each of the embodiments, the insert device is preferably configured to expand to form a seal of the body canal, wherein it is placed, once the compression (e.g. provided by a coating or an insertion-sleeve) is removed. Further, it can be seen on all the figures, that the devices is shaped/configured such the diameter of the absorbent material at the cross-section of the stoma, upon removal, is substantially similar to the diameter of the stoma. This diameter is different from the diameter of the absorbent material as expanded in the non-compressed state.

FIGS. 3A-3F shows cross-sections of the embodiments shown in FIGS. 1A-1F. These cross-sections correspond to the compressed state of the insert device.

FIGS. 4A-4F shows cross-sections of the embodiments shown in FIGS. 2A-2F. These cross-sections correspond to the non-compressed state of the insert device.

FIG. 5A shows the absorbent insert ostomy device according to another embodiment of the present disclosure. This embodiment of the insert device comprises an elongated body, which is formed from a sheet that is folded to form a wave-like pattern of crests and troughs.

FIG. 5B shows the absorbent insert ostomy device according to another embodiment of the present disclosure. This embodiment of the insert device comprises an elongated body constituting a cylinder that is folded one or more times.

The cross-sections of FIGS. 3A-3F are all drawn to scale in regards to the ratio with the cross-sections of FIGS. 4A-4F, i.e. the ratio between the diameter (the first diameter) of the cross-section of FIG. 3A-F and the diameter (the second diameter) of the cross-section of FIG. 4A-F, respectively, are correct. As exemplified, FIG. 3A has a cross-section where the diameter is approximate 2 cm (corresponding to the first diameter in the compressed state), whereas FIG. 4A has a cross-section where the diameter is approximately 4 cm (corresponding to the second diameter in the non-compressed state). This gives a ratio of 1 to 2 between the first and the second diameter of said embodiment. Similar calculation can be made based on the scale of the figures comparing 3B with 4B, 3C with 4C and so forth.

The absorbing organs shown in FIG. 1A-F has been compressed radially to a diameter of 1 to 3 cm, preferably produced in different sizes to suit the variable sizes of intestinal openings. The absorbing organs shown in FIG. 1A-C has been compressed longitudinally to a length of 5 to 20 cm (FIG. 1A-C). The absorbing organs shown in FIG. 1 D-F has not been compressed longitudinally and has the same length upon insertion as in their maximal absorbing state (FIG. 2 D-F). All absorbing organs (FIG. 1A-F) are held in the compressed state by itself, after compression, by an insertion device or by a thin cover (or wrapping) 5 of a material that is disintegrable by water, intestinal fluids, or heat. Soon after the insertion into the intestine, the insertion device will be withdrawn or the cover 5 will dissolve or disintegrate, whereby the absorbing material expands upon absorption to a size limited by the design and dimensions of the absorbing organs (seen in FIG. 3A-C). FIG. 2A-F show that the absorbing organs in the expanded state has a pre-defined size and shape whereby a too high pressure on the intestinal wall 6 is avoided. The absorbing organ can be held within the intestine by means of the radial expansion of the organ when not affected by the compressing factor, whereby the diameter of the organ exceeds the size of the intestinal opening 7 (FIG. 2A-F).

Upon extraction, the absorbing organs can be extracted through an intestinal opening 7 of minimum 1 cm. To ensure the capability of safe extraction, the dimension of the absorbing organ upon extraction does not exceed the dimension of the compressed absorbing organ upon insertion.

Under compression of the absorbing organ, the cover or insertion device may be placed on it, e.g. by wrapping with PVA film in a single layer with a small overlapping at which the PVA film adheres to itself by a modest moistening. The mounting may also take place by slipping a tubular film or insertion device over the organ.

FIG. 1-4 A shows an absorbing organ in the compressed (FIGS. 1 and 3) or expanded state (FIGS. 2 and 4). This organ has a spiral shape, and when it has reached its maximal absorption capacity (FIG. 2A), it has a diameter of 2 to 3 cm and a length of 15 to 40 cm in the spiral-folded state. The diameter of the strand forming the spiral is 1 to 1.5 cm, whereby the spiral can be extracted through an intestinal opening 7 of minimum 1 cm. In the distal end of the spiral it has a withdrawal means 9 (here a string) string attached that is partly left outside the intestinal opening 7 upon insertion. This string is used to pull out the spiral in a long strand. The spiral shaped absorbing organ may consist, entirely or partly of a hydrophilic polymer foam, e.g. polyurethane foam which may be prepared from a mould or by cutting from a bigger block. It could also consist entirely or partly of nonwovens, carboxymethyl cellulose, alginate, super absorbent polymers, super absorbent fibres or plant fibres/polymers. For some of the materials it would be beneficial to build the spiral on a stiff spiral core 10 to keep the spiral shape. This core could be made of e.g. silicone, rubber, latex, polyethylene or polyvinyl chloride.

FIG. 1-4 B shows an absorbing organ in the compressed (FIGS. 1 and 3) or expanded state (FIGS. 2 and 4). This organ has the shape of a brush and when it has reached its maximal absorption capacity (FIG. 2 B), it has a diameter of 2 to 3 cm and a length of 15 to 40 cm. The hairs of the brush are fixated on a core string 12 that is either solid or tubular. In order to extract the brush more easily through an intestinal opening 7 of minimum 1 cm, the core string 12 of the brush should preferable be resilient or elastic, so that the radial diameter of the brush is lowered upon extraction. A resilient or elastic core string 12 could be manufactured of e.g. silicone, rubber, latex, polyethylene or polyvinyl chloride. A stretchable core string 12 could also be a woven crinoline tube in materials as e.g. nylon, polyester, PVDF or polypropylene. The hairs of the brush may consist, entirely or partly of a hydrophilic polymer foam, e.g. polyurethane foam or nonwovens, carboxymethyl cellulose, alginate, super absorbent polymers, super absorbent fibres or plant fibres/polymers. In the distal end of the brush it has a withdrawal means 9 (here a string) attached that is partly left outside the intestinal opening 7 upon insertion. This string is used to pull out the brush and can be made of e.g. cotton.

FIG. 1-4 C shows an absorbing organ in the compressed (FIGS. 1 and 3) or expanded state (FIGS. 2 and 4). This organ has the shape of a tube and when it has reached its maximal absorption capacity (FIG. 2 C), it has a diameter of 2 to 3 cm and a length of 15 to 40 cm. In order to extract the tube through an intestinal opening of minimum 1 cm, the tube should be expandable, so that the radial diameter of the tube is lowered upon extraction. The tube could be manufactured as a woven crinoline tube in materials as e.g. nylon, polyester, PVDF or polypropylene to keep a stiffness of the tube. The woven tube could be blended, coated or partly filled with absorbing material 13, 14, and 15 e.g. hydrophilic polymer foam, nonwovens, carboxymethyl cellulose, alginate, super absorbent polymers, super absorbent fibres or plant fibres/polymers. In the distal end of the tube it has a withdrawal means 9, 16 (here a string) attached that is partly left outside the intestinal opening upon insertion. This string is used to pull out the tube, and is made of e.g. cotton.

FIG. 1-4 D shows an absorbing organ in the compressed (FIGS. 1 and 3) or expanded state (FIGS. 2 and 4). This organ has a conus shape and when it has reached its maximal absorption capacity (FIG. 2 D), it has a largest diameter of 2 to 3 cm and a length of 15 to 40 cm. The absorbing conus is made of a rectangular sheet of absorbent material (e.g. 10×30 cm) that is rolled a bit skewed into a conus shape. Along one end of the sheet a withdrawal means 9, 16 is attached (here a string), that is partly left outside the intestinal opening 7 upon insertion. This string is used to pull out the conus. To extract the conus through an intestinal opening 7 of minimum 1 cm, the rolled sheet will unfold upon extraction whereby the sheet will be pulled out in one layer like a spiral. The conus may consist, entirely or partly of a hydrophilic polymer foam, e.g. polyurethane foam or nonwovens, carboxymethyl cellulose, alginate, super absorbent polymers, super absorbent fibres or plant fibres/polymers.

FIG. 1-4 E shows an absorbing organ in the compressed (FIGS. 1 and 3) or expanded state (FIGS. 2 and 4). This organ has the shape of a cylinder and when it has reached its maximal absorption capacity (FIG. 2 E), it has a largest diameter of 2 to 3 cm and a length of 15 to 40 cm. The absorbing cylinder is made of a long tube in absorbing fabric (e.g. 2.5×60 cm) closed in one end and folded one or two times inside itself, to a length ½ or ⅓ of the initial tube. In the closed end of the tube a withdrawal means 9 is attached (here a string), that is partly left outside the intestinal opening 7 upon insertion. When pulling the string the cylinder unfolds to the long tube, whereby it can be extracted through an intestinal opening 7 of minimum 1 cm. The cylinder may consist, entirely or partly of a hydrophilic polymer foam, e.g. polyurethane foam or nonwovens, carboxymethyl cellulose, alginate, super absorbent polymers, super absorbent fibres or plant fibres/polymers. To keep the longitudinal length of the cylinder, it may be necessary to make some stiffening longitudinal seams or strings in nylon, polyester, PVDF or polypropylene. FIG. 5 shows alternative folding options of this absorbing organ.

FIG. 1-4 F shows an absorbing organ in the compressed (FIGS. 1 and 3) or expanded state (FIGS. 2 and 4). This organ consists of three (or two or four) neighbouring cylinders and when they have reached their maximal absorption capacity (FIG. 2 F), the organ has a largest diameter of 2 to 3 cm and a length of 15 to 40 cm. The absorbing cylinders may consist, entirely or partly of a hydrophilic polymer foam, e.g. polyurethane foam or nonwovens, carboxymethyl cellulose, alginate, super absorbent polymers, super absorbent fibres or plant fibres/polymers. In the end of every single cylinder, withdrawal means 9 are attached (here strings), that is partly left outside the intestinal opening 7 upon insertion. When pulling a string, only one cylinder is extracted, whereby it can be extracted through an intestinal opening 7 of minimum 1 cm. Extraction will remove all cylinders one by one (when repeated). The absorbing cylinders may consist, entirely or partly of a hydrophilic polymer foam, e.g. polyurethane foam which may be prepared from a mould or by cutting from a bigger block or tube. It could also consist entirely or partly of nonwovens, carboxymethyl cellulose, alginate, super absorbent polymers, super absorbent fibres or plant fibres/polymers. For some of the materials it would be beneficial to build the cylinder on a stiff core 17 to keep the longitudinal shape. This core could be made of e.g. silicone, rubber, latex, polyethylene or polyvinyl chloride. Another way to keep the longitudinal stiffness of the cylinder, would be to make some longitudinal seams or strings in nylon, polyester, PVDF or polypropylene.

Combinations of the described absorbing organs is possible. For example, a brush formed as a spiral or a smaller size brush (FIG. 1-4 B) or tube (FIG. 1-4 C) used as the cylinders in FIG. 1-4 F.

FIG. 8 shows directions of the expandable elongated body 20 as disclosed herein. The figure shows the radial direction 21 in which the body is expanding when going from the compressed state to the non-compressed state. The figure further shows the longitudinal direction 22 in which the body preferably expands when going from the compressed state to the non-compressed state, and in which direction the body extents.

The compressed state is when the one or more absorbent materials are in a “dry” state, such as before absorption of liquid from the intestines and before removal of the compression means. This means that the dimension can be measured by placing the device in a fixated state (by one or more compression means) on a table and measuring the dimensions. The non-compressed state is when the one or more absorbent materials are fully “wetted” in liquid, such as after end absorption in the intestines. This means that the dimension can be measured by placing the device in a relaxed state on a table and measuring the dimensions after being fully wetted.

Further shown is how the diameter 23 (the first and the second diameter) should be measured. Lastly the figure shows the oral end 25 and the anal end 24, to which the withdrawal means 9 are attached in this embodiment.

FIG. 7A shows any of the described embodiments, as disclosed herein, wherein the expandable elongated body 30 is in its compressed state. The body 30 is attached to a withdrawal mean 26 in the form of a string. Around the body 30 is wrapped a cover 29, which maintain the body 30 in its compressed state until the cover 29 is removed. The cover could e.g. be a PVA film, which would dissolve, when the device is inserted into the intestines.

FIG. 7B shows the absorbent insert ostomy device according to one embodiment of the present disclosure. This embodiment of the insert device comprises an elongated body 30, which is curled around a longitudinal axis of the body to form a helix comprising a plurality of turns. Further, the insert device comprises an elongated body with a plurality of flexible protrusions extending radially from the body 30, such that the elongated body 30 comprises a plurality of hair-like structures 31. The embodiment of 7B could effectively be seen as a combination of the embodiment of 1A and 1B, with slight modifications. This further highlight that combinations of the described absorbing organs are possible.

The embodiment of FIG. 7B is further configured to expand to form a seal of the body canal, wherein it is placed, once the compression (e.g. provided by a coating or an insertion-sleeve) is removed.

Upon extraction, the absorbing organs can be extracted through an intestinal opening of minimum 1 cm. To ensure the capability of safe extraction, the dimension of the absorbing organ upon extraction does not exceed the dimension of the compressed absorbing organ upon insertion. FIG. 7C shows how the device could look after extraction, where it can be seen how the elongated body 30 is further stretched, such that the winding of the spiral core 27 extends. This will happen when the device is extracted by pulling the withdrawal means 26.

The embodiment of FIG. 7B shows a device, preferably comprised of at least four components, an absorbent material, such as sodium carboxymethylcellulose (CMC) dressing, an elastic band 26 formed from e.g. elastane and polyester, a thread for sewing 28, such as e.g. a polyester thread, and a film to coat the absorbent material, such as e.g. a PVA film.

The device may then be produced as follows: Two identical bands of the absorbent material are placed on top of each other, both having a length of 50-100 cm and a width of 2.5-4.0 cm. Parallel cuts are made in the whole length of the bands, from both sides perpendicular to the longitudinal direction, leaving a central spine that holds the bands together and creates the so-called “hairs”, shown in the figure as hair-like structures 31. The two bands are then sewn together along the central spine 28, with an elastic band 26 that is stretched to 80-100% of maximal stretching capacity during the sewing process. The elastic band 26 is attached to the full length of the bands and continues a further 10-30 cm from where the bands end, leaving a solitary elastic band string to be used as a withdrawal string 26. Upon relaxation of the elastic band, the bands curl up into a spiral-shaped device 27 of approximately 20-35 cm in length with an appendix 26 of approximately 10-30 cm of the elastic band without absorbent material. The device is then compressed into a length of 3-8 cm and a diameter of 0.5-1.8 cm, onto which a PVA film is wrapped, permanently fixating the device in the compressed state. When the compressed device is inserted into the stoma, the secretion of the stoma causes the PVA film to dissolve. The absorbent body 30 then slowly expands in length and width as it absorbs the liquid faeces. The spiral shape 27 enables the tampon to function as a leak-tight (or semi-leak-tight) closure, by having a diameter of approximately 3 cm which is bigger than a typical stoma opening of 1.5 cm, whereas the tampon is kept in place by the abdominal wall when the peristaltic movements push it towards the abdominal wall. The small hair-like structures 31 collect solid parts of food in the faeces, which can then be withdrawn from the intestine together with the liquid faeces. They also ensure a large surface area of the absorbing material ensuring a large absorption capacity.

FIG. 8A shows the absorbent insert ostomy device according to one embodiment of the present disclosure. This figure illustrates the insert device when it is in a compressed state and when it is placed in the body canal of a subject. This embodiment of the insert device comprises an elongated body, which is curled around a longitudinal axis of the body to form a helix comprising a plurality of turns. This embodiment of the insert device further comprises the elongated body with a plurality of flexible protrusions extending radially from the body, such that the elongated body additionally resembles a brush.

FIG. 8B show a non-compressed state of the insert device according to the corresponding (compressed) embodiment of FIG. 8A. For the embodiments, the insert device is preferably configured to expand to form a seal of the body canal, wherein it is placed, once the compression (e.g. provided by a coating or an insertion-sleeve) is removed. Further, it can be seen on the figure, that the device is shaped/configured such the diameter of the absorbent material at the cross-section of the stoma, upon removal, is substantially similar to the diameter of the stoma. This diameter is different from the diameter of the absorbent material as expanded in the non-compressed state.

The absorbing organ shown in FIG. 8A has been compressed radially to a diameter of 1 to 3 cm, preferably produced in different sizes to suit the variable sizes of intestinal openings. The absorbing organ shown in FIG. 8A has been compressed longitudinally to a length of 5 to 20 cm. The absorbing organ is held in the compressed state by itself, after compression, by an insertion device or by a thin cover (or wrapping) of a material that is disintegrable by water, intestinal fluids, or heat. Soon after the insertion into the intestine, the insertion device will be withdrawn or the cover will dissolve or disintegrate, whereby the absorbing material expands upon absorption to a size limited by the design and dimensions of the absorbing organ (seen in FIG. 8B). FIG. 8A shows that the absorbing organ in the expanded state has a pre-defined size and shape whereby a too high pressure on the intestinal wall 6 is avoided. The absorbing organ can be held within the intestine by means of the radial expansion of the organ when not affected by the compressing factor, whereby the diameter of the organ exceeds the size of the intestinal opening 7.

Upon extraction, the absorbing organ can be extracted through an intestinal opening 7 of minimum 1 cm. To ensure the capability of safe extraction, the dimension of the absorbing organ upon extraction does not exceed the dimension of the compressed absorbing organ upon insertion.

Under compression of the absorbing organ, the cover or insertion device may be placed on it, e.g. by wrapping with PVA film in a single layer with a small overlapping at which the PVA film adheres to itself by a modest moistening. The mounting may also take place by slipping a tubular film or insertion device over the organ.

FIG. 8A-B shows an absorbing organ in the compressed (FIG. 8A) or expanded state (FIG. 8B). This organ has a spiral shape, and when it has reached its maximal absorption capacity (FIG. 8B), it has a diameter of 2 to 3 cm and a length of 15 to 40 cm in the spiral-folded state. The diameter of the strand forming the spiral is 1 to 1.5 cm, whereby the spiral can be extracted through an intestinal opening 7 of minimum 1 cm. In the distal end of the spiral it has a withdrawal means 9 (here a string) attached that is partly left outside the intestinal opening 7 upon insertion. This string is used to pull out the spiral in a long strand. The spiral shaped absorbing organ may consist, entirely or partly of a hydrophilic polymer foam, e.g. polyurethane foam which may be prepared from a mould or by cutting from a bigger block. It could also consist entirely or partly of nonwovens, carboxymethyl cellulose, alginate, super absorbent polymers, super absorbent fibres or plant fibres/polymers. For some of the materials it would be beneficial to build the spiral on a stiff spiral core to keep the spiral shape. This core could be made of e.g. silicone, rubber, latex, polyethylene or polyvinyl chloride. The shown absorbing organ further has on the outside of the spiral shape a brush shape, such that when it has reached its maximal absorption capacity (FIG. 8B), it has a diameter of 2 to 3 cm and a length of 15 to 40 cm. The hairs of the brush are fixated on either the core string or extend outside the helical core. In order to extract the brush more easily through an intestinal opening 7 of minimum 1 cm, the core string of the brush should preferable be resilient or elastic, so that the radial diameter of the brush is lowered upon extraction. A resilient or elastic core string could be manufactured of e.g. silicone, rubber, latex, polyethylene, or polyvinyl chloride. A stretchable core string could also be a woven crinoline tube in materials as e.g. nylon, polyester, PVDF or polypropylene. The hairs of the brush may consist, entirely or partly of a hydrophilic polymer foam, e.g. polyurethane foam or nonwovens, carboxymethyl cellulose, alginate, super absorbent polymers, super absorbent fibres or plant fibres/polymers.

### REFERENCES

5, 29—Cover
6—Intestinal wall
7—Intestinal opening
9, 16, 26—Withdrawal means
10, 27—Spiral core
12, 28—Core string
13, 14, 15—Absorbing material
17—Stiff core
20, 30—Expandable elongated body
21—Radial direction
22—Longitudinal direction
23—Diameter (e.g. first and second)
24—Anal end
25—Oral end
31—hair-like structure

The present invention is further illustrated by the following examples, which are not to be construed as limiting the scope of protection. The features disclosed in the foregoing description and in the following examples may, both separately or in any combination thereof, be material for realising the invention in diverse forms thereof.

### EXAMPLES

### Example 1—CMC Absorption Capacity of Water

To test the absorption capacity of a device as described herein towards water, a CMC material (100% carboxymethyl cellulose) piece was placed on a scale and the weight was noted (0.380 g). The piece of material was then placed in a petri dish and fully covered with water (tap water). The material was kept in the petri dish with water for 4 minutes after which the piece of material was pulled out of the petri dish with a tweezer and allowed to drip for 5 seconds, after which the material was weight on the scale (13.450 g). The result showed an absorption capacity of approximately 34 g of water per gram of CMC material.

### Example 2—CMC Absorption Capacity of Liquid Intestinal Waste

To test the absorption capacity of a device as described herein towards liquid waste from the intestines, and to see if similar absorption as towards water is possible, three CMC material (100% carboxymethyl cellulose) pieces was placed on a scale and the weight was noted (all weight 0.380 g). The pieces of material were then placed in three petri dishes and fully covered with liquid intestinal waste. The pieces were kept in the petri dishes with liquid for 4 minutes after which the pieces of material were pulled out of the petri dishes with a tweezer and allowed to drip for 5 seconds, after which each piece of material was weight on the scale (13.650 g; 13.123 g; 12.227 g). The result showed an absorption capacity of approximately 31 to 35 g of liquid per gram of CMC material, which corresponds to the absorption capacity of water.

1. An absorbent insert ostomy device for insertion through an ostomy of a human being, the absorbent insert ostomy device being configured to absorb intestinal fluid and waste inside the intestines of the human being, the absorbent insert ostomy device comprising:
an expandable elongated body extending along a longitudinal direction of the absorbent insert ostomy device and comprising a stoma end and an oral end spaced from the stoma end along the longitudinal direction, wherein the expandable elongated body comprises an absorbent material, wherein the absorbent material comprises carboxymethyl cellulose;
a withdrawer attached to the stoma end of the expandable elongated body; and
a compress configured to maintain the expandable elongated body in a compressed state, comprising a first diameter along the longitudinal direction, prior to insertion of the absorbent insert ostomy device through the ostomy of the human being;
wherein the expandable elongated body is configured to expand radially to a non-compressed state, comprising a second diameter along the longitudinal direction, when released from the compress upon insertion into an intestine of the human being through the ostomy;
wherein the second diameter is larger than the first diameter; and
wherein the expandable elongated body comprising the absorbent material is further configured to be pulled out of the intestine of the human being by the withdrawer.

2. The absorbent insert ostomy device according to claim 1, wherein the expandable elongated body changes from being rigid to being flexible when the expandable elongated body expands radially from the compressed state to the non-compressed state.

3. The absorbent insert ostomy device according to claim 1, wherein the absorbent material is biodegradable.

4. The absorbent insert ostomy device according to claim 1, wherein the absorbent material comprises at least 50% by weight of carboxymethyl cellulose compared to a total weight of the expandable elongated body.

5. The absorbent insert ostomy device according to claim 1, wherein the first diameter is 1.75 cm or less and the second diameter is at least 2 cm, wherein the expandable elongated body in the non-compressed state comprises a length along the longitudinal direction of at least 20 cm, and wherein the absorbent material comprises an absorption capacity of at least 50 ml.

6. (canceled)

7. (canceled)

8. The absorbent insert ostomy device according to claim 1, wherein the absorbent material comprises a modified cellulose.

9. (canceled)

10. (canceled)
